(19) United States
(12) Patent Application Publication
(10) Pub. No.: US 2026/0248388 A1
(43) Pub. Date: Aug. 27, 2026

(54) EYE EXAMINATION DEVICE AND METHOD FOR EYE EXAMINATION

(71) Applicant: Hooke Eye Exam Solutions Ltd., Or-Yehuda (IL)

(72) Inventors: Tal Zvi MARKUS, Kfar-Truman (IL); Eyal CAPUA, Tel Aviv (IL)

(73) Assignee: Hooke Eye Exam Solutions Ltd., Or-Yehuda (IL)

(21) Appl. No.: 18/870,705

(22) PCT Filed: Jun. 1, 2023
(86) PCT No.: PCT/IL2023/050569

§ 371 Date: Dec. 2, 2024

Related U.S. Application Data
(60) Provisional application No. 63/347,719, filed on Jun. 1, 2022.

(51) Int. Cl. A61B 3/18; A61B 3/00; A61B 3/032; A61B 3/103

(52) U.S. Cl. CPC A61B 3/18; A61B 3/0041; A61B 3/032; A61B 3/103

(57) ABSTRACT

Eye examination device combining objective and subjective optical examination subsystems, wherein the subjective exam is based on retinal scanning display technology and includes optical elements shared in common with the optical system for the objective exam. Potentially assists performing a fast visual acuity and/or refractive error exam, both objective and subjective, in a compact, simple, and potentially cost-efficient system. Exam results may provide a suitable basis for an eyeglasses and/or contact lens prescription.

### RELATED APPLICATIONS

This application claims the benefit of priority under 35 USC § 119 (e) of U.S. Provisional Patent Application No. 63/347,719 filed Jun. 1, 2022, the contents of which are incorporated herein by reference in their entirety.

### FIELD AND BACKGROUND OF THE INVENTION

The present invention, in some embodiments thereof, relates to the field of vision testing, and more particularly, but not exclusively, to visual acuity examinations.

Visual acuity examinations are usually comprised of an objective refractive error exam followed by a subjective visual acuity exam. The objective refractive error exam type device is commonly an auto-refractor or similar. The subjective visual acuity exam type device is most often a phoropter, through which the subject views a visual acuity chart (Snellen, Landolt rings, tumbling E). The visual acuity chart is usually placed at an apparent distance of about 6 meters for the far visual acuity test, or about 0.4 meters for the near visual acuity test.

Background art includes U.S. Pat. No. 4,465,348, U.S. Patent Application No. 2020/0077885, and U.S. Patent Application No. 2020/0275833.

### SUMMARY OF THE INVENTION

According to an aspect of some embodiments of the invention, there is provided an eye examination device which operates to perform both subjective and objective visual acuity testing, including, during testing, projection of images and test patterns onto at least a first retina of a tested subject, the device including: a first optical pathway, including a retinal scanning display that projects a subjective visual acuity testing image onto the first retina; a second optical pathway, configured to project an objective visual acuity testing pattern onto the first retina, and including a sensor which detects light returning from the first retina from the objective visual acuity testing pattern; wherein the retinal scanning display projects a target image onto the first retina as a target for visual fixation and/or accommodation relief by the subject while the second optical pathway operates to project the objective visual acuity testing pattern.

According to some embodiments of the invention, the eye examination device includes optics in the first optical pathway which adjust to introduce varying optical power to a first image beam which impinges on the first retina to produce the subjective visual acuity testing image.

According to some embodiments of the invention, the optics comprise at least one spherical correction element which adjusts to introduce varying spherical optical power to the first image beam.

According to some embodiments of the invention, the optics comprise at least one cylinder correction lens which adjusts to introduce varying cylindrical optical power and a varying cylindrical optical axis to the first image beam.

According to some embodiments of the invention, the cylinder correction lens includes at least one of the group consisting of a liquid lens and a liquid crystal lens.

According to some embodiments of the invention, the at least one cylinder correction lens includes a first cylinder correction lens group and a second cylinder correction lens group; each group including at least one cylindrical lens; wherein the first and second cylinder correction lens groups: have cylindrical optical powers of opposite signs; adjust together to introduce the varying cylindrical axis while maintaining mutual alignment of their respective cylindrical axes; and adjust by changing their relative distance to each other to introduce the varying cylindrical optical power.

According to some embodiments of the invention, the mutual alignment of the respective cylindrical axes orients them within 5° of each other.

According to some embodiments of the invention, the eye examination device includes: a first projection system for the first retina, including the first optical pathway, second optical pathway, and retinal scanning display; a second projection system for a second retina of the tested subject, including features corresponding to each respective feature recited for the first projection system; and wherein the first and second projection systems operate together to provide respective images to the first and second retinas arranged as a binocularly registered image.

According to some embodiments of the invention, the eye examination device includes: one or more mechanical degree of freedom stages allowing positioning optical pupils of the first and second projection systems in relative positions accommodating a range of eye placement geometries for human subjects; and at least one sensor configured to detect a state of binocular eye alignment of the optical pupils to eyes of the subject at least during preparation for testing of the tested subject.

According to some embodiments of the invention, the eye examination device includes: one or more actuators to move the one or more stages; a controller including a processor configured to operate the one or more actuators according to the detected state of binocular eye alignment to align the optical pupils to the eyes of the tested subject.

According to some embodiments of the invention, at least one of the first and second projection systems includes a moving element which adjusts relative viewing angles of their respective first optical pathways.

According to some embodiments of the invention, the moving element is actuatable to adjust the relative angle during presentation of at least one of the subjective visual acuity testing image and the target image.

According to some embodiments of the invention, actuation of the moving element simultaneously adjusts relative viewing angles of the respective first and second optical pathways.

According to some embodiments of the invention, the eye examination device includes, for each of the first and second projection systems, a respective third optical pathway, configured to merge a view of the surroundings of the tested subject with any of the images and testing pattern of the first and second optical pathways.

According to some embodiments of the invention, the eye examination device includes at least one eye tracking device configured to track at least one of eye state and eye position during one or both of subjective visual acuity testing and objective visual acuity testing.

According to some embodiments of the invention, the subjective visual acuity testing image includes at least one of the group consisting of: a Snellen chart, a Landolt ring chart, a tumbling E chart, Lea test, HDTV chart, sunburst chart, clock dial chart, and a spatial frequency chart.

According to some embodiments of the invention, the subjective visual acuity testing image is presented binocularly with depth cues locating at least two portions of the subjective visual acuity testing image to different apparent distances from the subject.

According to some embodiments of the invention, the objective visual acuity testing pattern includes a plurality of beams selected to indicate refractive error by impinging on the retina differently for different refractive errors of eye optics focusing light on the retina according to the Scheiner principle.

According to some embodiments of the invention, the eye examination device includes a processor configured to receive data from the sensor, and determine refractive error of the eye optics based on at least one of the group consisting of: Shack-Hartmann wavefront sensing, knife edge effect, ray tracing aberrometry, image size principle, and/or the Scheiner principle.

According to some embodiments of the invention, the objective visual acuity testing pattern includes a pattern of beams projected sequentially onto the retina; and including a processor which receives data from the sensor, and determines wave-front error according to correlations between directions of beams of the pattern entering the eye, and retro-reflections of the pattern leaving the eye and detected by the sensor.

According to some embodiments of the invention, the retinal scanning display of the first optical pathway includes a MEMS mirror, and the MEMS mirror is also used in production of the objective visual acuity testing pattern.

According to some embodiments of the invention, the retinal scanning display of the first optical pathway includes a plurality of MEMS mirrors, which operate to scan one or more beams along different axes.

According to some embodiments of the invention, the eye examination device includes a keratometer.

According to some embodiments of the invention, the target image includes at least one of a blurred region and a moving object moving in apparent depth.

According to an aspect of some embodiments of the invention, there is provided a method of eye examination, including: aligning an optometric examination device to the eyes of a subject, and while the device remains aligned to the eyes of the subject: projecting a subjective visual acuity testing image onto a first retina using a retinal scanning display of the device; projecting an objective visual acuity testing pattern onto the first retina using an illumination source of the device; detecting light returning from the first retina from the objective visual acuity testing pattern using a light sensor of the device; and projecting, using the retinal scanning display, a target image onto the first retina as a target for visual fixation and/or accommodation relief by the subject during the projecting and detecting of the objective visual acuity testing pattern.

According to some embodiments of the invention, the method includes adjusting optical correcting power used to project the subjective visual acuity testing image onto the first retina, based on input from the subject.

According to some embodiments of the invention, the subjective visual acuity testing image is presented binocularly, and the input from the subject includes an indication of the comparative appearance of the binocularly presented image among two eyes.

According to some embodiments of the invention, the method includes projecting the target image onto the first retina through optics adjusted while projecting the subjective visual acuity testing image onto the first retina.

According to some embodiments of the invention, the method includes performing each of the operations performed on the first retina also on a second retina of the subject, and also while the optometric examination device remains aligned to the eyes of the subject.

According to some embodiments of the invention, at least one of the subjective visual acuity testing image and target image is presented to the subject binocularly.

According to some embodiments of the invention, the method includes adjusting an eye vergence of the subject by adjusting an apparent depth of the binocularly presented at least one image.

According to some embodiments of the invention, the method includes adjusting a lens accommodation of the subject by adjusting an apparent depth of the binocularly presented at least one image.

According to an aspect of some embodiments of the invention, there is provided an image display device, including: an optical pathway configured to project an image onto a retina of an eye, and including cylindrical lenses which introduce varying cylindrical optical power and a varying cylindrical optical axis to beams impinging on the retina to produce the image; wherein the cylindrical lenses comprise a first cylinder correction lens group and a second cylinder correction lens group, each group including at least one cylindrical lens; and wherein the first and second cylinder correction lens groups: have cylindrical optical powers of opposite signs; adjust together to introduce the varying cylindrical axis while maintaining a predetermined relative alignment of their respective cylindrical axes; and adjust by changing their distance to each other to introduce the varying cylindrical optical power.

According to some embodiments of the invention, at least the second cylinder correction lens group includes a plurality of cylindrical lenses; and cylinder optical powers of the plurality of cylindrical lenses combine along the optical pathway to produce the cylindrical optical power of opposite sign to the first cylinder correction lens group.

According to some embodiments of the invention, the second cylinder correction lens group includes at least one lens on either side of at least one lens of the first cylinder correction lens group.

According to some embodiments of the invention, at least one lens of the second cylinder correction lens group moves along the optical pathway to vary the introduced cylindrical optical power, and there is at least one position of the at least one lens of the second cylinder correction lens group which cancels cylindrical optical power of the first cylinder correction lens group.

According to some embodiments of the invention, the first and second cylinder correction lens groups are positioned within a telecentric region of the beams.

According to some embodiments of the invention, the beams have an overall envelope diameter, and are individually imaged to focal positions on the retina; and the envelope diameter of the beams is approximately constant between the first and second cylinder correction lens groups.

According to some embodiments of the invention, the individual beams each have a respective beam waist within a region defined between the lenses of first and second cylinder correction lens groups.

According to some embodiments of the invention, for the first and second cylinder correction lens groups adjust their distance to each other to change cylindrical correction power through a range of at least 2 diopters.

According to some embodiments of the invention, the first and second cylinder correction lens groups rotate together around an optical axis of the optical pathway to introduce the varying cylindrical axis.

According to some embodiments of the invention, the image display device forms an optical pupil to the eye having a first diameter in a direction wherein the diameter is maximally affected by the varying cylindrical optical power, and a second diameter orthogonal to the first diameter; and throughout a range of at least four diopters of adjustment, a ratio of the first and second diameters remains less than 2.

According to some embodiments of the invention, the display illumination includes at least one of the group consisting of: a μLED display, a μOLED display, LED display, an OLED display, a QDLED display, an LCD display, and LCOS source, a DLP source, and a scanned beam source.

According to an aspect of some embodiments of the invention, there is provided a method of varying cylindrical aberration introduced within an image-forming light beam, the method including: moving a first cylindrical lens from a first position to a second position along an optical axis of the light beam; wherein: moving the first cylindrical lens changes a distance to a second cylindrical lens, each of the first and second cylindrical lenses has a cylindrical axis, and the respective cylindrical axes are mutually aligned; wherein the beam, beyond the first and second cylindrical lenses, forms an optical pupil having a first diameter in a direction maximally affected by the movement of the first cylindrical lens, and a second diameter orthogonal to the first diameter; and throughout a range of at least four diopters of cylindrical aberration introduced to the light beam, a ratio of the first and second diameters remains less than 2.

According to some embodiments of the invention, during the moving, for at least one position of the first cylindrical lens, cylindrical power introduced to the beam after passing the first and second cylindrical lenses is substantially zero.

According to some embodiments of the invention, the second diameter is less than 5 mm.

According to an aspect of some embodiments of the invention, there is provided an eye examination device, including: a first optical pathway, configured to project a subjective visual acuity testing image onto a retina of a tested subject; a second optical pathway, configured to project an objective visual acuity testing pattern onto the retina of the tested subject, and including a sensor configured to sense light returning from the retina from the objective visual acuity testing pattern; wherein the first optical pathway projects a target image onto the retina as a target for visual fixation and/or accommodation relief by the subject while the second optical pathway operates to project the objective visual acuity testing pattern; and a third optical pathway, configured to merge a view of the surroundings of the tested subject with any of the images and testing pattern of the first and second optical pathways.

According to some embodiments of the invention, the eye examination device includes optics in the first optical pathway which adjust to introduce varying optical power to a first image beam which impinges on the first retina to produce the subjective visual acuity testing image.

According to some embodiments of the invention, the optics comprise at least one spherical correction element which adjusts to introduce varying spherical optical power to the first image beam.

According to some embodiments of the invention, the optics comprise at least one cylinder correction lens which adjusts to introduce varying cylindrical optical power and a varying cylindrical optical axis to the first image beam.

According to some embodiments of the invention, the cylinder correction lens includes at least one of the group consisting of a liquid lens and a liquid crystal lens.

According to some embodiments of the invention, the at least one cylinder correction lens includes a first cylinder correction lens group and a second cylinder correction lens group; each group including at least one cylindrical lens; wherein the first and second cylinder correction lens groups: have cylindrical optical powers of opposite signs; adjust together to introduce the varying cylindrical axis while maintaining mutual alignment of their respective cylindrical axes; and adjust by changing their relative distance to each other to introduce the varying cylindrical optical power.

According to some embodiments of the invention, the mutual alignment of the respective cylindrical axes orients them within 5° of each other.

According to some embodiments of the invention, the eye examination device includes: a first projection system for the first retina, including the first, second, and third optical pathways; a second projection system for a second retina of the tested subject, including features corresponding to each respective feature recited for the first projection system; and wherein the first and second projection systems operate together to provide respective images to the first and second retinas arranged as a binocularly registered image.

According to some embodiments of the invention, the eye examination device includes: one or more mechanical degrees of freedom stages allowing positioning optical pupils of the first and second projection systems in relative positions accommodating a range of eye placement geometries for human subjects; and at least one sensor configured to detect a state of binocular eye alignment of the optical pupils to eyes of the subject at least during preparation for testing of the tested subject.

According to some embodiments of the invention, the eye examination device includes: one or more actuators to move the one or more stages; a controller including a processor configured to operate the one or more actuators according to the detected state of binocular eye alignment to align the optical pupils to the eyes of the tested subject.

According to some embodiments of the invention, at least one of the first and second projection systems includes a moving element which adjusts relative viewing angles of their respective first optical pathways.

According to some embodiments of the invention, the moving element is actuatable to adjust the relative angle during presentation of at least one of the subjective visual acuity testing image and the target image.

According to some embodiments of the invention, actuation of the moving element simultaneously adjusts relative viewing angles of the respective first and second optical pathways.

According to some embodiments of the invention, the eye examination device includes at least one eye tracking device configured to track at least one of eye state and eye position during one or both of subjective visual acuity testing and objective visual acuity testing. According to some embodiments of the invention, the subjective visual acuity testing image includes at least one of the group consisting of: a Snellen chart, a Landolt ring chart, a tumbling E chart, Lea test, HDTV chart, sunburst chart, clock dial chart, and a spatial frequency chart.

According to some embodiments of the invention, the subjective visual acuity testing image is presented binocularly with depth cues locating at least two portions of the subjective visual acuity testing image to different apparent distances from the subject.

According to some embodiments of the invention, the objective visual acuity testing pattern includes a plurality of beams selected to indicate refractive error by impinging on the retina differently for different refractive errors of eye optics focusing light on the retina according to the Scheiner principle.

According to some embodiments of the invention, the eye examination device includes a processor configured to receive data from the sensor, and determine refractive error of the eye optics based on at least one of the group consisting of: Shack-Hartmann wavefront sensing, knife edge effect, ray tracing aberrometry, image size principle, and/or the Scheiner principle.

According to some embodiments of the invention, the objective visual acuity testing pattern includes a predetermined pattern of beams projected sequentially onto the retina; and including a processor which receives data from the sensor, and determines wave-front error according to correlations between directions of beams of the predetermined pattern entering the eye, and retro-reflections of the pattern leaving the eye and detected by the sensor.

According to some embodiments of the invention, the eye examination device includes a keratometer.

According to some embodiments of the invention, the target image includes at least one of a blurred region and a moving element moving in apparent depth.

According to some embodiments of the invention, the image display device comprises display illumination including at least one of the group consisting of: a μLED display, a μOLED display, LED display, an OLED display, a QDLED display, an LCD display, and LCOS source, a DLP source, and a scanned beam source.

According to an aspect of some embodiments of the invention, there is provided a method of performing a visual acuity exam, including: in a portion of an open retail space defined by one or more display cabinets and a countertop, providing an optometric acuity testing device accessible to test subject at the countertop, or in a space bounded on two or more sides by at least one of or a combination of the countertop and the one or more display cabinets; operating the testing device to perform both subjective and objective acuity testing while a subject remains aligned thereto; and providing a lens prescription to the subject, based on results of the acuity testing.

According to some embodiments of the invention, the method includes recording an order for vision correcting optics from the subject, the vision correcting optics including at least one article of sale chosen according to articles displayed in the one or more display cabinets.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method, or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Instruction executing elements of the processor may comprise, for example, one or more microprocessor chips, ASICs, and/or FPGAs. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable medium to receive instructions and/or data therefrom, process them, and/or store processing results in the same or another computer readable medium. The processing performed (optionally on the data) is specified by the instructions, with the effect that the processor operates according to the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object-oriented programming language such as Java, Smalltalk, C++ or the like and conventional procedural programming languages, such as the “C” programming language or similar programming languages. Additionally or alternatively, sequences of logical operations (optionally logical operations corresponding to computer instructions) may be embedded in the design of an ASIC and/or in the configuration of an FPGA device. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer; and may not be feasible or practical for performing purely manually, by a human expert. A human expert who wanted to manually perform similar tasks, such inspecting objects, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1A schematically illustrates a cylindrical refractive error correction unit, according to some embodiments of the present disclosure.

FIG. 1B schematically illustrates a cylindrical refractive error correction unit, according to some embodiments of the present disclosure.

FIGS. 2A-2B schematically illustrate pupil effects of the introduction of cylindrical correction in one- and two-lens cylindrical correction units, according to some embodiments of the present disclosure;

FIGS. 3A-3B schematically illustrates an optical system comprising subjective and objective testing optical subsystems, according to some embodiments of the present disclosure;

FIG. 4 schematically illustrates an optical system providing a separate objective testing illumination source, according to some embodiments of the present disclosure;

FIG. 5A schematically illustrates a cylindrical refractive error correction unit in combination with a spherical refractive error correction unit, according to some embodiments of the present disclosure;

FIG. 5B schematically illustrates a cylindrical refractive error correction unit in combination with a screen illumination source, according to some embodiments of the present disclosure;

FIG. 6 schematically illustrates a block diagram of an optical system combining objective and subjective eye refraction testing subsystems, according to some embodiments of the present disclosure;

FIG. 7 schematically illustrates a block diagram of a variant of optical system combining objective and subjective eye refraction testing subsystems, and having a dual-use scanning unit, according to some embodiments of the present disclosure;

FIG. 8 schematically illustrates a block diagram illustrating optical modules of a variant of optical system combining objective and subjective eye refraction testing subsystems, and having a dual-use scanning unit, according to some embodiments of the present disclosure;

FIG. 9 schematically illustrates a block diagram illustrating optical modules of a variant of optical system combining objective and subjective eye refraction testing subsystems, having a dual-use scanning unit, according to some embodiments of the present disclosure;

FIG. 10 is a schematic optical diagram of the objective testing optical subsystem of optical system of FIG. 9, according to some embodiments of the present disclosure;

FIG. 11 is a schematic optical diagram of the Scheiner principle, performed, for example, using the optical arrangements of FIG. 10, according to some embodiments of the present disclosure;

FIG. 12 is a schematic optical diagram of a subjective testing optical subsystem, according to some embodiments of the present disclosure;

FIG. 13 is a schematic optical diagram of combined objective and subjective testing optical subsystems of an optical system, according to some embodiments of the present disclosure;

FIG. 14 is a schematic optical diagram of combined objective and subjective testing optical subsystems of an optical system using two scanning units, according to some embodiments of the present disclosure;

FIG. 15 is a schematic optical diagram of combined objective and subjective testing optical subsystems of an optical system, with the objective testing optical subsystem using ray tracing aberrometry, according to some embodiments of the present disclosure;

FIG. 16 is a schematic flowchart of a method for measuring the refractive error of subject's eyes and providing a prescription for eyeglasses or contact lenses, according to some embodiments of the present disclosure;

FIG. 17 is a schematic flowchart of a method to align a subject's eye to an optical system, according to some embodiments of the present disclosure;

FIG. 18 schematically illustrates a compact visual acuity and/or refractive error testing system, according to some embodiments of the present disclosure;

FIGS. 19A-19B schematically represent table-top use of compact visual acuity testing kit to perform visual acuity testing on a subject, according to some embodiments of the present disclosure;

FIG. 19C schematically illustrates a refractive error examination system installed in a kiosk stand, according to some embodiments of the present disclosure; and

FIGS. 20A-20C schematically illustrate head-worn implementations of a vision testing system, according to some embodiments of the present disclosure;

### DESCRIPTION OF SPECIFIC EMBODIMENTS OF THE INVENTION

The present invention, in some embodiments thereof, relates to the field of vision testing, and more particularly, but not exclusively, to visual acuity examinations.

### Overview

A broad aspect of some embodiments of the present disclosure relates to vision testing systems and/or methods configured for both objective and subjective measurement of eye refraction (an important factor in visual acuity); for example, to determine corrective lens prescriptions for eyeglasses and/or contact lenses. The measurements and/or determination of an associated corrective lens prescription comprise, in some embodiments, determination of refractive error (quantifying eye refraction) and/or visual acuity (a measure of the spatial resolution of the visual processing system overall, determined subjectively, and affected by eye refraction).

In some embodiments, an objective testing optical subsystem comprises at least one beam source, and optics that direct light from the at least one beam source to the subject's eye, along one or more optical pathways. In some embodiments, the optical pathway includes a scanning mirror (e.g., a microelectromechanical system [MEMS] scanning mirror), configured to direct incident light from the at least one beam source to different locations (e.g., via adjustment of the angle of the mirror). This is used, for example, to produce light patterns appropriate for testing, which project onto the subject's eye. In some embodiments, the mirror is operated at a scanning speed producing a frame rate greater than 30 Hz, preferably larger than 50 Hz. The resolution is preferably suitable to produce, e.g., 800×600, 1280×720 or a higher pixel resolution image. After added optics, this may be used to produce an image with at least about 1 arcminute, 0.5 arcminutes, or 0.3 arcminutes of resolution (e.g., a resolution sufficient to allow detection of human eye optical acuity loss within ordinarily accepted limits of optical acuity testing). Optionally, the angular size of the image subtends, for example, about ±5° horizontally (e.g., at least ±3°), and about ±3° vertically (e.g., at least)±2°.

In some embodiments, light from a first beam source reflected from a surface of the eye is directed to a sensor and analyzed. The sensed reflected light carries refractive information, used in the analysis to estimate the objective refraction of the subject's eye. Light emitted from the first beam source may be in the near infrared (IR), e.g., a wavelength greater than 640 nm. Optionally or additionally, light of another wavelength is used.

During an objective visual acuity exam, the subject focuses on a target. The target optically comprises a blurred region, e.g., a blurred background region, and optically an element moving in apparent depth, e.g., moving away from the viewing subject. In some embodiments, the scanning mirror is used also to create the target that the subject focuses on, e.g., a target formed from light emitted by a second beam source. In some embodiments, the second beam source, scanning mirror and optical pathway used for creating the target in the objective visual acuity exam are also used in the subjective visual acuity exam for presenting visual acuity charts and/or other images.

In some embodiments, a subjective testing optical subsystem is configured for determining the refraction of a subject's eye subjectively, based on subject reporting of the appearance of test images produced by a projection unit. The images, in some embodiments, are projected onto the retina of the subject by scanning one or more laser beams from a beam source (e.g., the second beam source) in two dimensions. Scanning is produced, e.g., by adjustment of a scanning mirror angle. Optionally, the same mirror is used in both subjective and objective testing.

In some embodiments, the subjective testing optical subsystem includes a defocusing assembly unit, acting as the phoroptor of the device to apply adjustable corrective refractive power comprising at least a spherical correction applied to the image the subject sees. The defocusing unit is adjustable to produce step and/or continuous changes in the divergence or convergence of the laser beam. Subject feedback is used to determine the refractive power which best corrects the subject's vision.

The subjective testing optical subsystem optionally includes a cylindrical and axis optical correction assembly. This introduces an adjustable distortion to the shape of the scanned laser beam. During acuity testing, adjustment is selected to counteract the cylindrical and axis error of the subject's eyes, e.g., based on feedback from the subject reporting how clearly a target is seen, and/or what target is seen and/or distinguishable. In some embodiments, the subsystem includes an optical relay, configured to provide magnification as appropriate, and bring the exit pupil of the optical subsystem to the subject's eye. When the second beam source is used to present the focus target presented together with the objective visual acuity exam, the defocusing assembly unit and cylindrical and axis optical correction assembly optical subsystem may be present in or removed from the optical path. If present, they are optionally adjusted to neutral settings (e.g., neutral positions), or to positions which are known and/or estimated to fully or partially correct refractive error of the eyes of the subject.

In some embodiments, a starting point which sets shaping of the scanned beam in the subjective visual acuity exam is based on available data indicative of the subject's refractive error. The data comprise, for example, the subject's previous prescription, measurements of currently used prescription lenses, and/or any other available information related to a subject's vision or eye medical history. Additionally or alternatively, the initial configuration is based on data obtained from an objective visual acuity exam, e.g., an exam performed using the objective testing optical subsystem. The objective testing optical subsystem optionally makes use of any appropriate objective lens characterizing effect, principle, sensor, and/or method, for example, Shack-Hartmann wavefront sensing, knife edge effect, image size principle, ray tracing aberrometry, and/or Scheiner principle.

The initial configuration is optionally set exactly according to the best available data or offset from it, e.g., with an added or subtracted factor. For example, about 2-4 diopters (e.g., 2, 3, or 4 diopters) of positive or negative offset is applied to reduce accommodation. Based on feedback from the subject, the spherical, cylindrical and axis are adjusted, until their combined correction produces the best-seen image on the subject's retina. From parameters which characterize the changes to the scanned beam (e.g., divergence/convergence, astigmatism and cylindrical axis) which produce the optimized image on the subject's retina, the subjective spherical, cylindrical and axis error are determined, providing information for an eyeglasses or contact lenses prescription, optionally in conjunction with additional information such as corneal curvature.

Optionally, one or both of the objective and subjective testing optical subsystems is duplicated (e.g., copied in at least their major optical features, optionally with packaging adjustments such as mirror imaging of component layout), allowing both eyes to be tested simultaneously or alternately without adjusting the position of the system. Preferably, images produced by the duplicate systems on are binocularly registered to produce a perceptually unified image for the subject (assuming the subject is capable of depth perception within the range of physical settings available to the device). This optionally produces a sense of depth in the image. Optionally, the binocularly registered image includes differences between images, comprising, for example, depth cues and/or feature differences used in testing.

Optionally, one or both of the objective and subjective testing optical subsystems can be shunted alternately to either eye (e.g., using a mirror), and/or is visible simultaneously to each eye (e.g., using a prism and/or beam splitter. For example, in some embodiments, objective acuity testing occurs at least partially concurrently with subjective acuity testing. For example, objective visual acuity testing may make an incidental use of a subjective visual acuity test target as the attention-drawing stimulus which helps to set the subject's lens accommodation. It is emphasized in particular with regard to this concurrency that objective visual testing optionally uses IR (invisible) wavelengths for the testing itself, and optionally shares the same display for target presentation as is used during subjective visual acuity testing.

Optionally, intermediate results of one testing modality are used to adjust testing of the other modality, and optionally iteratively. For example, objective visual testing may be used to set an initial range of subjective visual testing corrective power to test, while results of the subjective testing may then be used to adjust the presentation of visual stimuli which help set the accommodation of the subject for further objective visual acuity testing, and/or to adjust the range of test parameters within which further objective testing is concentrated. Insofar as the same device performs both functions, such mutual reinforcement of results is potentially enabled without a requirement to reposition the subject/device, or even to perceptibly interrupt one test type in order to perform the other.

An aspect of some embodiments of the present disclosure relates to providing capabilities in a combined objective and subjective visual acuity testing device with internal display, which said capabilities promote the guidance and/or control of subject attention, eye vergence (inter-eye difference in angle of viewing), and/or lens accommodation. In some embodiments, the capabilities promote the combination of the visual acuity testing device with other devices making use of external screens, e.g., devices for teleconferencing and/or auxiliary visual testing. In some embodiments, capabilities are provided which allow enhancements to visual acuity testing relating to speed, comfort, accuracy, and/or automaticity of testing.

In some embodiments, images are delivered simultaneously to both eyes. This potentially promotes using the testing device to influence the state of the subject's eyes by manipulation of factors including, for example:
Inter-pupil distance (IPD), which can be changed mechanically, e.g., by moving the exit pupils of device optics further or closer to each other in a horizontal direction. Optionally, a system is configured with degrees of freedom to adapt mechanically to other aspects of a subject's anatomy as well; e.g., to account for relative vertical displacement and/or relative depth displacement of the subject's eyes relative to the facing plane.
Eye vergence, which can be changed, e.g., by altering the angle of mechanical and/or optical elements of the system, e.g., mirrors and/or prisms (though the latter is liable to introduce chromatic aberration). Optionally, the whole optical subsystem for an eye is moved, which provides the potential advantage of maintaining a constant optic axis once the eye itself adjusts position to th new angle. For example, an eye vergence of about 1° is typical for viewing a target at a range of about 3-6 meters, while an eye vergence of about 3° is typical for viewing a target at a range of about 0.4 meters.

It is noted that particularly when the maximum visual field size of presented images is relatively small (e.g., ±5°), the use of mechanical movements of the system to adjust eye vergence may help preserve substantially the whole area available for visual presentation. In some embodiments, eye vergence is adjusted (additionally or alternatively) by displacing the generated images left/right within the available visual field size of the retinal scanning display (i.e., displacing the images digitally). Mechanical adjust went of eye vergence provides the potential advantage (compared to digital image displacement) of maintaining a constant angle to the eye, as the eye angle moves to follow the new angular position. For example, it potentially maintains an angle at which an objective visual acuity test pattern is projected onto the retina.

In some embodiments, a combined-testing device is configured to internally generate and present test and/or target images to one or both eyes of a subject. In some embodiments, the generated images are optionally delivered to the eye(s) through a beam which is also combined with light collected from in front of the subject, so that the subject sees the generated images combined with an optically formed image of their surroundings (e.g., an augmented reality or AR view). This potentially assists in setting and/or controlling the lens accommodation of the subject. For example, arbitrary objects (e.g., an attention-drawing objects such as a toy; actually present in the environment and/or shown as 3-D images) may be shown to a child subject to draw their attention through the passthrough image collected from the surroundings. Optionally, a person familiar to such a subject can draw the subject's attention from in front of the subject via the passthrough image.

The objects of the surroundings in any case potentially assist by providing a familiar visual context to the subject's visual system. The passthrough image also potentially promotes interaction between the subject and the eye examiner (optometrist) and/or technician (assistant), since the subject's view is then not necessarily isolated to what is generated within the testing device.

In some embodiments, brightness of the generated images is adjustable (e.g., by adjusting laser power, polarizers, filters, and/or LC filters) to conduct the vision acuity exam under targeted conditions (e.g., as standardized, common, or optimal according to the preferences of the test administrator(s)). Optionally, the brightness of the surroundings is adjusted, e.g., using an adjustable polarizer, transparent LCD, or other device. This has potential advantages for allowing the subject to comfortably view the test targets in a wide range of ambient illumination conditions, so that use can be made of the subject's natural optical accommodation responses to the positions of objects in actual surroundings.

In some embodiments, passthrough of the surroundings promotes combination of the image presentation capabilities built into the testing device with other displays such as teleconferencing displays and/or special testing displays which may be separately available in an examination setting. For example, the subject can interact with (e.g., take instructions from/ask questions to) a test examiner visible on a teleconferencing screen without necessarily interrupting visual acuity testing and/or positioning with respect to a visual acuity testing device. The external screen may be used to present images which draw the attention of the subject, e.g., in place of a toy or other attention-attracting object.

In some embodiments, a visual acuity testing device includes supporting functions such as eye tracking, which optionally includes, more specifically, one or more of eye position (e.g., eye vergence) tracking, eye lens accommodation tracking, and/or pupil size tracking. Together with the passthrough capability, this allows optional use of the device together with an external screen which, e.g., presents stimuli for auxiliary assessment of vision, visual attention, or another purpose. With or without the use and/or presence of passthrough capability, eye tracking functions are optionally used internally to assess the validity of test results (e.g., objective visual acuity testing) during different trials. Optionally, loss of correct eye positioning is used to trigger corrective and/or alerting measures, e.g., repositioning of device optical pupils, alteration of presented visual stimuli, discarding of bad data, and/or presentation of a warning to the device operator.

In some embodiments, presentation of images to both eyes or just one eye is varied as appropriate to the type and/or phase of testing. For example, appropriate optical correction to achieve good visual acuity for a subject may be determined alternately in each eye, and then the two eyes stimulated together using a binocularly registered image, e.g., in such a way as to allow the subject to judge if both eyes see equally well, or if one of them is noticeably under-corrected compared to the other. For example, a test image may be broken into zones of features which together form a single image, at least some of which are presented only to one eye, and/or one eye at a time.

In some embodiments, lens power adjustments are under automatic control which allows automatic testing to be

An aspect of some embodiments of the present disclosure relates to providing cylindrical and axis correction to the optics of a subjective and/or objective visual acuity testing device. In some embodiments, the visual acuity testing device presents images using a scanning display device.

In some embodiments, the cylindrical and axis correction is provided together with a vision testing system configured for one or both of objective and subjective measurement of eye refraction; for example, to determine corrective lens prescriptions for eyeglasses and/or contact lenses. In some embodiments, a cylindrical refractive error correction unit is provided as an element of a virtual reality (VR) and/or augmented reality (AR) system.

A retinal scanning display (also known as a virtual retinal display, retinal scan display, or retinal projector) projects images onto the retina by the projection of suitably patterned light. Rather than relaying a conjugate image of a light source (e.g., a light source patterned with the scene of the image), such a display builds an image on the retina directly, by scanning a light source (e.g., one or more lasers) across the retina's surface in a rapid, intensity modulated pattern. In effect, light from the beam source(s) is optically manipulated within the display so that it reaches the entrance pupil of the eye from different directions, rather than the single (generally fixed) each beam source actually occupies.

Accordingly, information about different angularly defined regions of the image scene is conveyed through beams arriving to the eye from those different angles. The image builds up over a period of time, but quickly enough that a single image is perceived. From whatever direction it arrives, the scanned beam reaches the entrance pupil of the eye. For design purposes, the entrance pupil is optionally considered as a circle or ellipse varying in the range of 2-6 mm in diameter, for example, about 2 mm, 3 mm, 4 mm, 5 mm, or 6 mm; though an actual eye entrance pupil is potentially smaller (e.g., in high lighting conditions) or larger (e.g., if medically dilated); e.g., in a range of 2-8 mm or 2-10 mm.

The cornea and lens focus light entering the eye's pupil from a single beam to a spot on the retina. Accordingly, production of such images is affected by optics of the eye itself in the usual ways, including spherical refraction error and cylindrical aberrations, such as are routinely corrected for using eyeglasses and contact lenses.

Although a retinal scanning display may internally create a “scanned beam” (the beam comprising all angles of the laser beam over the course of an image frame), if optical power corrections are applied to counteract flaws in the optics of the eye, they are applied chiefly to the laser beam itself, however it may be directed at any given moment. Put another way, the laser beam at each moment corresponds to a “pixel” which should be brought to optimal focus on the retina. In short, optical corrections are applied “intra-beam”.

Spherical error and/or cylindrical aberration are commonly corrected using personalized (optically static) corrective lenses. However, the use of certain optical systems (e.g., optometric testing systems) relies on being able to provide a range of such corrections. Potentially, certain virtual reality (VR) and/or augmented reality (AR) systems and/or users may benefit from such capabilities.

Concentrating on cylindrical correction, a basic arrangement for introducing an adjustable optical power and/or axis direction of cylindrical distortion to an optical system's wavefront may rely simply on providing a plurality of cylindrically distorting lenses of different powers, with a lens of appropriate power being selected to replace any other lens in the system, e.g., manually, or by movement of a carousel. Cylinder axis may be selected in such a system by rotating the lens around the optical axis.

Another arrangement for introducing adjustable optical power and/or axis direction of cylindrical distortion comprises a plurality of lenses (e.g., a pair of lenses), arranged in directions which cross each other to a variable extent. For example, one lens may introduce negative optical power, and the other positive optical power. If each lens has the same magnitude, then when they are aligned there is minimal cylindrical power introduced (e.g., none of practical significance). When they are orthogonally placed, maximal cylindrical distortion is introduced. Intermediate levels of cylindrical distortion are introduced by intermediately oblique crossing angles. For a given selected cylindrical power, the cylinder axis direction may then be selected by rotating the crossed cylindrical lenses together by the same amount.

Accordingly, the crossing lenses may be positioned in an optical path to act effectively as a single variable-power cylindrical lens, providing a potential advantage by reducing a need to swap optical components.

However, arrangements to introduce cylindrical distortion to an optical system potentially interact with spherical correction, in a manner which negatively affects calibrations and/or focusing of beams to pupils of the optical system. This may be the case particularly when the system is intended to function with one or both of spherical power and cylindrical power being varied. However, the inventors of the present disclosure have determined the existence of a class of optical arrangements which can reduce this issue sufficiently to potentially remove it as a limiting factor on overall device performance, while providing potential advantages for compactness and/or simplicity of device design and/or function.

Briefly, “vergence” is a property of a beam comprising non-parallel light rays. For beams overall (in geometrical optics, e.g., not necessarily considering wavelength dependent effects), vergence is defined in terms of the curvature of the beam's optical wavefront, expressed as optical power (e.g., in diopters with a unit of m−1). With increasing distance from an unmodified beam's source, vergence approaches zero (flatness), similar to the way the local curvature of a sphere approaches flatness as the sphere gets larger and larger. Lenses in the optical path introduce changes to the wavefront curvature. For example, they may collimate it (reducing the vergence to near zero, or parallel), increase it, or decrease it. This can include switching vergence sign (e.g., switching a diverging beam to a converging beam). One convention for schematically depicting a beam along an optical path draws a path of at least one of its outer (marginal) rays. In such cases, the changing angle of the marginal ray(s) relative to the optical axis may be used as the measure of vergence.

Scanned beam optical systems such as retinal scanning displays produce “superimposed” vergences—one describing the wavefront curvature of an individual laser beam position (herein, the intra-beam vergence), and one describing the wavefront curvature of the collection of scanned beam positions (herein, the inter-beam vergence). In scanned beam optical systems, the presence of more than one vergence imposes design constraints affecting the beam's condition at entrance and/or exit pupils—locations in the optical path which image the (physical) aperture stop. In a system intended to illuminate the retina of an eye, the relevant aperture stop which these pupils image may be the eye's own (anatomical) pupil. Without suitable management of both laser beam (intra-beam) and scanned beam (inter-beam) vergences to converge at an illuminating optical system's entrance pupil(s), illumination and/or some of the field of view is lost, degrading the beam that is actually able to enter the eye's entrance pupil to form an image on the retina.

There is, in particular, a potential problem of introducing cylindrical correction intra-beam, without disturbing inter-beam relationships (e.g., without preventing focusing at the entrance pupil). For example, a cylindrical lens (including an arrangement of two or lenses which is or “acts as” as an adjustable cylindrical lens) can be placed in the optical path and create cylindrical correction effects on intra-beam vergence. But in many locations (e.g., locations away from an inter-beam pupil), this will also produce inter-beam vergence changes.

In some embodiments of the present invention, cylindrical correction is applied to the illumination beam of a retinal scanning display by placing two lens groups, each of one or more cylindrical lenses, in the optical path of the illumination beam. In some embodiments, the first and second lens groups are arranged to have (and preserve) a same shared cylinder axis, which in some embodiments is changeable (e.g., by rotation).

This allows differential effects on inter-beam vs intra-beam vergences to be produced. In some embodiments, the differential effects amount to a negligible change in inter-beam cylindrical vergence, but a significant (e.g., astigmatism-correcting) level of change in intra-beam cylindrical vergence. Since, in this case, inter-beam vergence magnitude is relatively small relative to overall beam width, differences in effects as a function of distance along the optical path are relatively negligible. For example, inter-beam vergence changes introduced by the first group encountered on the beam path can be effectively canceled by the second-encountered group (which may be of opposite diopter sign). To achieve cancellation, the two groups may have optical powers with the same diopter magnitude, or only slightly different. Optionally, the difference is selected to correct for the relatively small effects the of distance offset.

Accordingly, in some embodiments, one group is placed closer to an intra-beam waist and one group is further. Positions and strengths of the two lens groups are selected so that intra-beam vergence changes induced by the further of the cylindrical lens groups dominate (are larger than) vergence changes induced by the closer of the cylindrical lenses to introduce cylindrical distortion, but inter-beam vergence changes induced by the two groups is substantially diminished.

In some embodiments, the amount by which one lens group dominates the other is selectable to produce a magnitude of overall intrabeam cylinder vergence change reaching at least −8, −6, −5, −4, −3, or −2, negative diopters, and/or at least +2, +3, +4, +5, +6, or +8 positive diopters. Optionally, the same lenses are adjustable in position so that the magnitude of intrabeam cylinder vergence is continuously changed, or changed with steps at least as small as, for example, about 0.1, 0.125, 0.2, or 0.25 diopters. Optionally, at least 10 different magnitudes of overall intrabeam vergence change are selectable between maximum and minimum magnitudes. In some embodiments, both positive and negative overall vergence changes (e.g., in diopters) are selectable. In some embodiments, the overall selectable range in diopter change is at least 2, 4, 6, 8, 10, 12, or 16 diopters, including both positive and negative diopters.

In some embodiments, the first and second groups of one or more cylindrical lenses are of opposite diopter signs. Furthermore, in some embodiments, they are of similar magnitudes, e.g., similar within 50%, 25%, 10%, or 5%. In some embodiments, the first and second groups of one or more cylindrical lenses are placed on opposite sides of an intra-beam waist. Optical power may be divided among two or more lenses in one or both groups. The lenses within a group are optionally be arranged along an optical path section (e.g., a telecentric zone) so that together they approximate the effects of a single lens (a “virtual lens”) occupying a position between them.

In some embodiments of the present invention, cylindrical correction is applied using a system having the following features:
At least two cylindrical lenses are provided.
These lenses are configured to produce a net effect on intrabeam vergence, variable within a practical range of astigmatism encountered in optometry (e.g., −2 to 2 diopters, −3 to 3 diopters, −4 to 4 diopters, −6 to 6, or −8 to 8 diopters).
However, there is also sufficient opposition of optical powers of the cylindrical lenses that net effects on inter-beam vergence are negated, at least to the extent that focusing light to the eye's entrance pupil sufficient to display a complete test image is not significantly compromised.

In some embodiments, accordingly, there are provided a first at least one cylindrical lens and a second at least one cylindrical lens, wherein the at first and second cylindrical lenses (or lens groups) impose opposing vergence effects on the inter-beam vergence.

Regarding the phrase “not significantly compromised” used above with respect to pupil formation; “significant compromise” is optionally evaluated functionally, according to the performance of a subject undergoing testing for visual acuity. The performance should, in this case, not be adversely affected (compared to an actual or extrapolated “perfect” pupil). For example subject performance is not measurably, distinguishably, and adversely affected in terms of test accuracy, testing speed and/or (self-reported) subject comfort. Potential causes of performance degradation consequent to poor optical pupil formation (should it occur) include, for example: vignetting (relative darkening, e.g., of image edges), partial loss of test target visualization, difficulty in perceiving presented test targets, and/or difficulty in distinguishing differences in test targets among different conditions.

in some embodiments, “not significantly compromised” is determined with respect to device parameters; the determination comprising, for example, one or more of the following criteria:
The angular size of the test images shown to a subject fully subtends a specified region of interest (both vertically and horizontally) extending over at least 1° 2°, 3°, or 10°. The region of interest may be defined, for example, according to the test target size and/or resolution.
The image display device may be considered as forming an optical pupil to the eye, typically by lens(es) that focus all beams to nearly the same location, each beam starting from approximately the same angle of incidence with the lens(es). In a system where the individual beams are almost parallel to each other or nearly parallel (for example a telecentric or nearly telecentric zone): after the converging lens, all beams will form the optical pupil at nearly the same position, with the diameter of the beams at this position defining the diameter of the optical pupil. When applying cylindrical power, the system can be viewed as having two optical pupils distributed along the optical axis, according to the effect of the cylindrical power on each axis. At one extreme is a first pupil is formed by beams shifted maximally by the varying cylindrical optical power. At the other is a second pupil formed by beams positioned where they are essentially unaffected by the chosen axis of cylindrical optical power—the “original” pupil position. If the positions of the two optical pupils do not sufficiently coincide, a larger-diameter pupil is then found at the “original” (without cylindrical corrective power) position of the optical pupil, since some beams (e.g., those most affected by the cylinder optical power) are displaced from their “home” pupil: fanning out from it, or yet to converge to it. Under these conditions, and throughout a range of optionally at least 2, 3, 4, 5 or more diopters of cylinder adjustment, a ratio of the optical pupil's diameters (that is, its longest and shortest diameters, respectively along directions with and without cylindrical optical power distortions) remains less than about, for example, 3, 2.5, 2, 1.5 or 1.1.
All parts of the test image region of interest provide maximal retinal illumination levels within at least 15% of a nominally sufficient average level for specified functioning of the device (e.g., visual testing, or other image presentation). Optionally, the threshold is at least 25%, 33% or 50%.
Particularly for applications of embodiments for which image brightness is limiting (i.e., applications where reducing maximum brightness to achieve overall evenness reduces an aspect of usability), the maximum brightness in the brightest illuminable regions is no more than 50% brighter than the maximum brightness in the dimmest illuminable regions. This may be understood as a measure of how much illumination compensation is needed to overcome flaws in optical pupil formation.

Disturbance of intra-beam vergence is potentially minimized by placing the cylindrical lens where the laser beam for each position of the larger scanned beam has its waist (that is, its narrowest point between two wider regions).

For example, when placing a cylindrical lens having focal length F₂ after a spherical lens of focal length F₁, the focus point changes in each axis according to the power of the lenses in that axis, and the distance between them. As the cylindrical lens has power in a single axis, that axis is modified; the other axis remains unaffected. Rotating the cylindrical lens allows choosing the affected axis.

Modifying the distance D between the lenses changes the focus point F_{T} of the affected axis in accordance with Equation 1:

$$\frac{1}{{F}_{T}} = \frac{1}{{F}_{1}} + \frac{1}{{F}_{2}} - \frac{D}{{F}_{1} {F}_{2}} \qquad Equation 1$$

The distance of the “new focus” from the second lens is the back focus length (BFL), described in Equation 2:

$$B F L = {F}_{2} ( D - {F}_{1} ) / ( D - ( {F}_{1} + {F}_{2} ) ) \qquad Equation 2$$

Accordingly: when the distance between the lenses (D) approaches focal length F₁, BFL=0. In this position the vergence of the beam is not affected; only its width.

If a third lens is placed after the focus point of the two-lens system described above at a distance from the focus point of the unaffected axis that is equal to its own focus length, the light beam will be collimated on that axis, but not on the axis chosen to position the cylindrical lens when BFL≠0. This allows modifying the vergence at a chosen axis by rotating the lens.

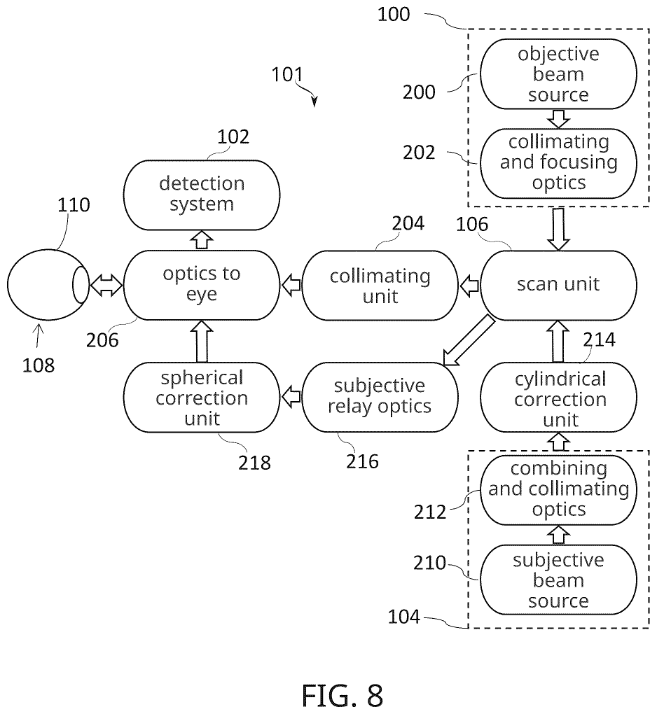

Cylinder correction introduced to the illumination path of a scanning display potentially enables its use in optometric testing of visual acuity and/or eye refractive error, for example, as detailed in relation to FIGS. 8-15, herein. In some embodiments, built-in cylinder correction may enable users of retinal scanning display devices used for general display purposes to obtain a clear image without necessarily having to use other corrective lenses (e.g., their usual eyewear), with potential benefits for device size and/or wearing comfort (e.g., less need to make room for wearing eyeglasses together with the device).

In some embodiments, cylindrical corrective lens is used which is intrinsically adjustable in optical power and/or axis (an adaptive lens); for example, a liquid lens and/or liquid crystal lens. Such a lens is optionally placed at an optical pupil of the optical system, where it potentially has a selective effect on intrabeam vergence. Embodiments herein are optionally used with an arrangement of this sort, insofar as they are not explicitly limited to use of another arrangement for providing cylindrical correction. For example, a cylindrical correction unit 214 generally may be implemented using such an arrangement (perhaps substituted for the arrangement of another implementation described herein), and optionally moved to a more suitable location in the optical path (e.g., an optical pupil) as appropriate. It is anticipated that during the lifetime of patent(s) potentially maturing from this application, new relevant forms of intrinsically adjustable lenses will be developed, including and/or other than those referred to herein as liquid lenses and/or liquid crystal lenses. The term “intrinsically adjustable lens” is intended to include all such new technologies a priori.

Although embodiments of the present disclosure are described in particular in relation to retinal scanning displays, it should be understood that other display types (particularly miniaturized display types) are optically used; e.g., replacing the retinal scanning display laser and MEMS mirror, and suitably coupled into the rest of the system using appropriate adaptor optics. A hallmark of a highly miniaturized display may be its ability to produce and form an image and/or image beam from a source contained within a volume having an optical cross-section (perpendicular to the optical path) of about 10 cm² or less, 6.5 cm² or less, 4 cm² or less, or 1 cm² or less. However, a less miniaturized display is optionally used in some embodiments of the present disclosure.

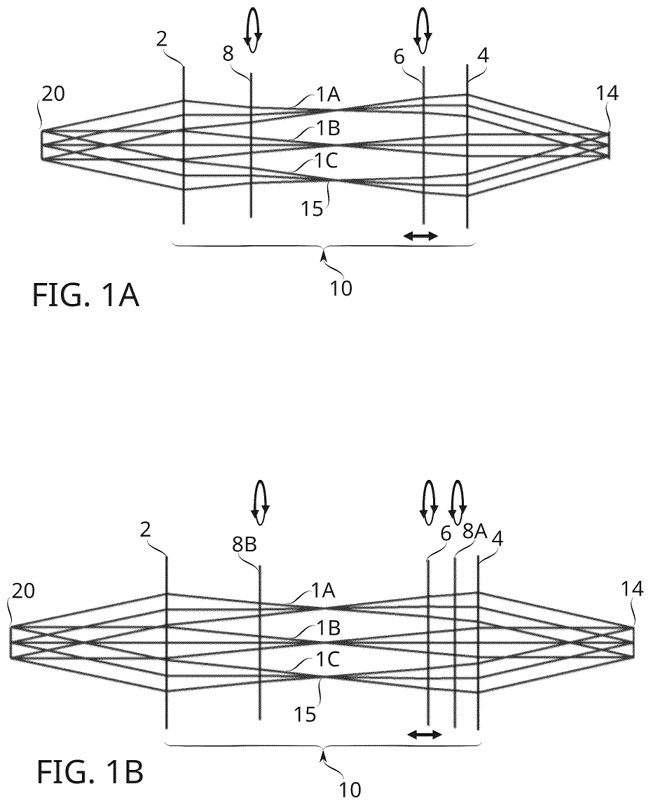

For example, in some embodiments, a micro LED (also known as mLED or μLED) display replaces one or more image beam generating elements in any of FIGS. 1A-20C, with adapting optics provided as appropriate. In some embodiments (e.g., embodiments implementing aspects of these figures), another display technology is used; for example, μOLED, LED, OLED, QDLED, LCD, LCOS, DLP, or another technology. It is anticipated that during the lifetime of patent(s) potentially maturing from this application, new relevant forms of miniaturized display technologies will be developed and/or become available on the market, including and/or other than those mentioned herein. The term “display” is intended to include all such new technologies a priori.

Before explaining at least one embodiment of the present disclosure in detail, it is to be understood that the present disclosure is not necessarily limited in its application to the details of construction and the arrangement of the components and/or methods set forth in the following description and/or illustrated in the drawings and/or given in the Examples. drawings. Features described in the current disclosure, including features of the invention, are capable of other embodiments or of being practiced or carried out in various ways.

### Cylindrical Correction

Reference is now made to FIG. 1A, which schematically illustrates a cylindrical refractive error correction unit, according to some embodiments of the present disclosure.

In some embodiments, a cylindrical lens arrangement comprises two cylindrical lenses 6, 8 within a telecentric zone 10 established by two lenses 2, 4. Individual beams, however, are not collimated in zone 10.

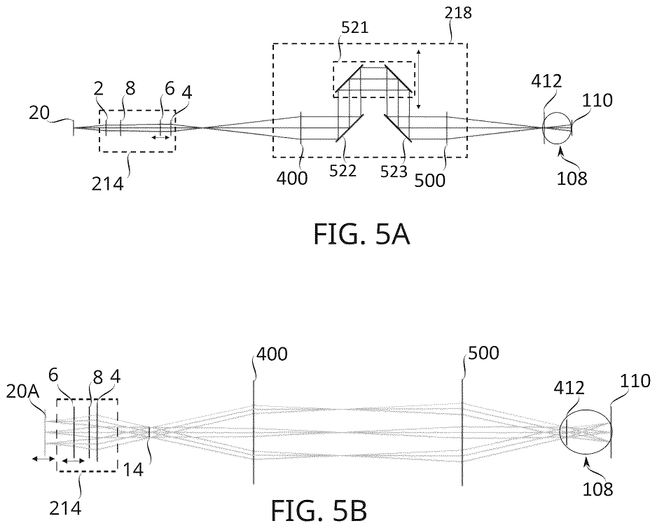

The image eventually projected onto by the retina is generated by light source 20 on the left side of the drawing. Implementation details of this light source are not shown in FIG. 1A, but may be implemented, e.g., as described in relation to any of FIGS. 8-15 herein, for example as a scanning retinal display, or another type of display (e.g., incorporating changes, for example as described in relation to FIG. 5B). Embodiments of FIG. 1A are optionally provided with embodiments of these figures, e.g., as implementations of a cylindrical correction unit 214. Beyond exit pupil position 14 at the right side of the drawing, the light is eventually relayed via further optics of the optometric testing device to the patient's own pupil and retina, e.g., to perform a subjective vision acuity test.

Selected individual laser beams 1A-1C are each drawn as three non-parallel lines (rays) representing the width (the outer or “marginal” rays) and centerline of the laser beam at different distances. Waist position 15 indicates the location of the narrowest region of the beams 1A-1C along the length of the telecentric zone. For the whole image to be seen by the patient, beams 1A-1C should overlap with the centerline beam within the area of the exit pupil at exit pupil position 14 (and also again at the patient's own pupil).

In some embodiments, light source 20 comprises a laser and one or more scanning mirrors. It operates by rapidly scanning laser beams in a 2-D pattern that angles outward to fill collimating lens 2. For purposes of explanation, the beams are depicted as arriving at angles which collimating lens 2 makes parallel, creating the telecentric zone 10. This could be varied somewhat to change the spherical corrective refractive power. Optionally, spherical correction is applied by separate optics, e.g., as described in relation to FIGS. 8-15 herein.

To provide variable orientation of cylindrical refraction, cylindrical lenses 6, 8 can rotate around the optical axis to change the orientation of the cylindrical correction. To provide variable refractive power, at least one of the cylindrical lenses 6, 8 (lens 6 in the embodiment shown) is moveable along the optical axis. This changes the corrective cylindrical refractive power applied to the image which the patient sees. With greater distance of a cylindrical lens from waist position 15, a larger correction is imposed. In some embodiments, cylindrical lenses 6, 8 are mutually aligned in their respective cylindrical axes; e.g., exactly aligned, or aligned at least to within 5°, 3°, or 1° of each other. These values in degrees should also be understood be available (without limitation) to other examples of mutually aligned cylindrical axes described herein, changed as appropriate, unless incompatible or otherwise stated.

As also discussed in the overview, providing a second cylindrical lens has potential advantages, because there are two different “vergences” which must be co-managed along the optical pathway. For example, at each position of its scanning pattern, the rays of a laser beam may begin approximately parallel (collimated, corresponding to a low intra-beam vergence). However, the beams at different positions of the scanning pattern are spread-angled compared to each other (diverging, e.g., “negative” inter-beam vergence). Both vergences, existing inside the same overall system, are changed in different ways by the lenses the beam encounters. In particular, the intra-beam vergence is not parallel (not collimated) in telecentric zone 10, as illustrated by the crossing lines defining each laser beam 1A-1C, including its waist position 15. The inter-beam vergence, in contrast, is parallel or nearly parallel.

The position of single cylindrical lens 6 in telecentric zone 10 modifies both vergences. Changing the intra-beam vergence gives the desired effects on optical power in a chosen axis. Changing the inter-beam vergence, however, has an effect on convergence at the exit pupil, which could prevent some of the beams from entering the subject's pupil.

By introducing counter-refracting lens 8 with a refractive power opposite to that of lens 6, the actual overall effect on inter-beam convergence is potentially greatly diminished. For example, lens 8 introduces non-parallel inter-beam vergence which is largely canceled a short distance later by lens 6. Beginning from this arrangement, if the distance through which lens 6 moves when modifying the cylindrical power is kept short, the exit pupil size at exit pupil position 14 will not change significantly; that is, near-cancellation of inter-beam vergence happens throughout a relatively large range of lens positions.

This allows the two rotationally aligned lenses 6, 8 to have nearly the same effect in positions throughout telecentric zone 10. In contrast, the lens effects on intra-beam vergence are highly dependent on distance from waist position 15, since moving a lens closer to waist position 15 proportionally weakens its effect on cylindrical correction.

In some embodiments, this module or unit for optical cylinder correction comprises, a device for introducing cylindrical correction to one or more scanned laser beams, comprising two rotatable cylindrical refractive units (e.g., single lenses, or groups of lenses) positioned in a telecentric zone of an optical system. At least one of the units is movable along an axis of the telecentric zone to produce a variable net effect on intra-beam vergence. The cylinder axes of the two units are similarly oriented, and have complementary optical powers selected to largely cancel out each other's effects on inter-beam vergence.

In some embodiments, the one or more scanned laser beams are used in a retinal scanning display system.

In some embodiments, moreover, the scanned laser beams are produced as part of the illumination system of an optometric device incorporating both objective and subjective refractive error visual acuity testing capabilities (e.g., testing for eyeglasses prescriptions). At least the subjective test makes use of the portion of the optical pathway incorporating the two cylindrical refractive units. For example, in a subjective test, the retinal scanning display produces a target image, and cylindrical correction is adjusted until the target is seen clearly according to patient reporting. In an objective test using the same device, the retinal scanning display may be used to produce a target image, which is used to guide the direction and distance of visual focus (subject accommodation), while optics in another part of the system provide test illumination and measurement functions.

It should be noted that the optical elements described from lens 2 to lens 4 (and in particular, the optical subsystem of lenses 6, 8) are operable with any suitable source of illumination, so long as lens 2 (or another arrangement of optics) interacts with the beam that arrives at it so as to generally maintain the position of the exit pupil of the system. For example, the magnitude of the inter-beam vergence is reduced sufficiently to allow differential introduction of cylinder correction, while preserving beam entrance to the pupil of the subject's eye. In some embodiments, this comprises the projected image which then reaches the retina being seen without disturbance to testing results, e.g., due to vignetting or loss of field of view of the projected image. The optical elements including lenses 6, 8 may be provided after a previous relay section of the optical path, after folding mirrors, after beam splitters, or a part of another suitable arrangement of optical elements which interact with the illumination beam.

Reference is now made to FIG. 1B, which schematically illustrates a cylindrical refractive error correction unit, according to some embodiments of the present disclosure.

Except as next noted, the elements shown in FIG. 1B generally correspond to elements of FIG. 1A, and embodiments according to FIG. 1B may be used to provide cylinder correction to optical systems also as described in relation to FIG. 1A.

In some embodiments, the net refractive power of cylindrical lens 8 (of FIG. 1A) is divided among two or more lenses 8A, 8B, one on either side of cylindrical lens 6. Lenses 8A, 8B are positioned to allow the overall net cylindrical refractive power (also including lens 6) to be changed from positive to negative diopters, optionally while moving a single lens. Dividing potentially enables this because with suitable selection of lenses, the “average” position of lenses 8A, 8B may be placed optically nearer to the middle of telecentric zone 10 than lens 8 is (e.g., on the right side of the waist position 15), although no physical lens is actually there. Accordingly, cylindrical lens 6 can be moved to either side of that position.

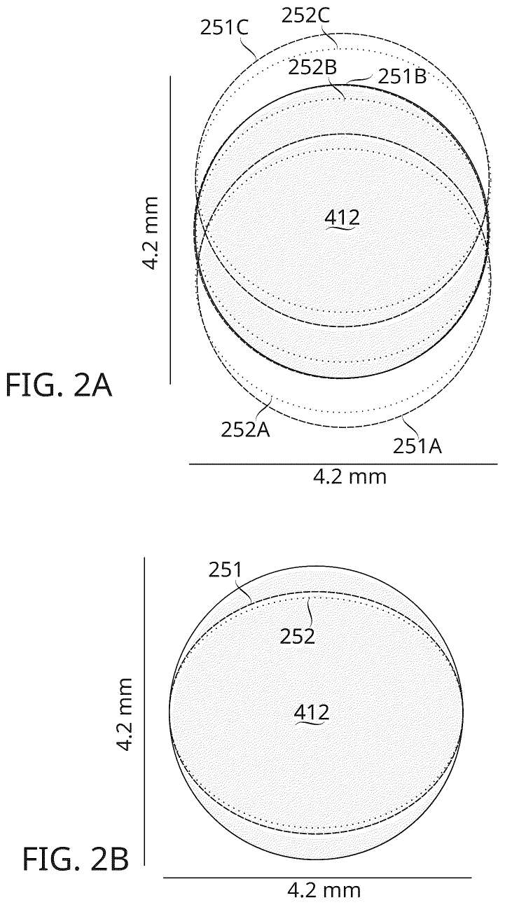

Reference is now made to FIGS. 2A-2B, which schematically illustrate pupil effects of the introduction of cylindrical correction in one- and two-lens cylindrical correction units, according to some embodiments of the present disclosure.

The pupil effects are simulated for optical systems defined as follows. In both of FIGS. 2A and 2B, shaded area represents a nominal 4 mm diameter pupil. In the examples shown, the system is designed for optimal pupil at a cylinder correction of −2 cylinders. Pupil dispersion (pupil size) results shown are for cylinder corrections on either side of this value by ±2 diopters. For the sake of illustration, a 1:1 relay is assumed. Effects on pupil dispersion potentially increase as the magnification power of the relay is increased, which is expected to be the case at least for a scanning retinal display. For example, in some embodiments, relay magnification in the range of 1:1 to 1:8 is used; e.g., a 1:1.5, 1:2, 1:3, 1:4 1:5, 1:8 or other relay magnification factor is used. These numbers optionally apply to any of the embodiments described herein.

In FIG. 2A, just a single cylinder lens is used to introduce cylindrical aberration to the wavefront (e.g., equivalent to lens 6 of FIG. 1A without providing lens 8), with a power of 8 diopters.

In FIG. 2B, two opposing-sign cylinder lenses are used to introduce cylindrical aberration to the wavefront (e.g., equivalent to both of lenses 6 and 8 of FIG. 1A), with powers of 8 diopters (moving) and −10 diopters (stationary).

Accordingly, regions 251A-251C (FIG. 2A) and region 251 (FIG. 2B) represent beams dispersal by the introduction of cylinder correction of −4 diopters. Regions 252A-252C (FIG. 2A) and region 252 (FIG. 2B) represent beam dispersal by the introduction of cylinder correction of 0 diopters.

In FIG. 2A, beam crescents falling outside of shaded area 412 (e.g., for regions 251A, 251C, 252A, 252C represent lost light, with consequent degradation of image quality.

In FIG. 2B, dispersals of the beams are indistinguishable, and so each condition is represented by one region (regions 251 and 252) only. There is no loss of light at the pupil due to pupil dispersion.

As mentioned, larger cylindrical corrections and/or larger magnifications by the relay will tend to exaggerate effects, e.g., to the point that some beams may fail to enter the pupil 412 at all in the single-cylinder correction mode. It may be noted that there is no horizontal dispersion, since cylindrical correction is only applied along one axis. Optionally, this allows the orthogonal axis to the axis of maximum dispersion to be used as a reference for evaluating the magnitude of pupil dispersion under various conditions.

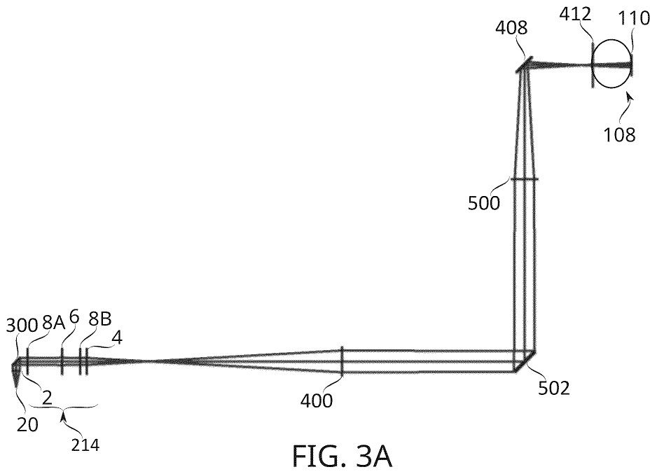

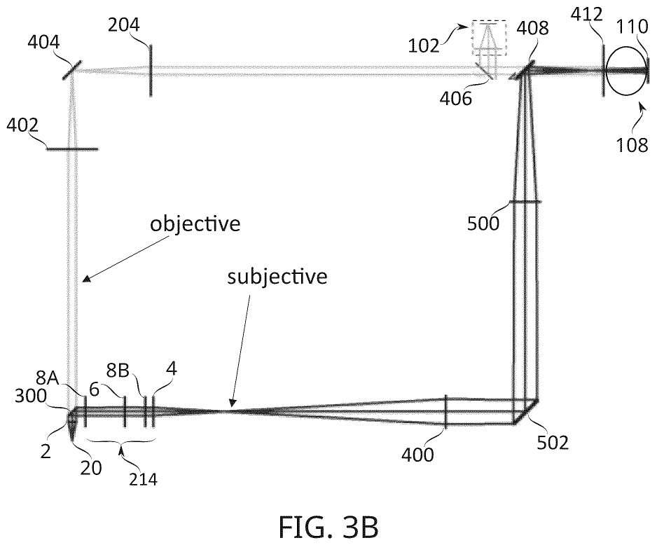

Reference is now made to FIGS. 3A-3B, which schematically illustrates an optical system 101 comprising subjective and objective testing optical subsystems, according to some embodiments of the present disclosure. In this embodiment, light source 20 also acts as an objective test illumination source 200. It is optically operated together with subjective pathway illumination activated for use as a focusing and/or accommodation relief target by the subject.

Elements 20, 2, 8A, 6, 8B, and 4 correspond to the designations shown for the adjustable cylinder correction unit of FIG. 1B, comprising a cylindrical correction unit 214, for example as described in relation to FIGS. 8-15, herein.

Beam splitting optics 300, lens 402, optional folding mirror 404, collimating optics 204, beam splitter 406, beam combiner 408, objective test detection unit 102, relay lens 400, folding mirror 502, relay lens 500 eye 108, eye pupil 412, and retina 110 correspond to same-numbered elements described in relation to FIGS. 8-15 herein; noting, however, that relay lens 400 is shown here after beam splitting optics 300, its role in collimating the objective light path being taken over by collimating lens 2 of the cylinder correction unit.

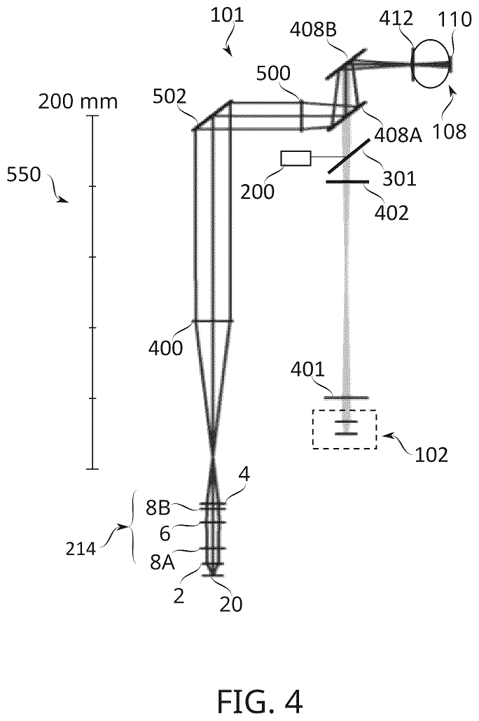

Reference is now made to FIG. 4, which schematically illustrates an optical system 101 providing a separate objective testing illumination source 200, according to some embodiments of the present disclosure.

Several elements of the subjective illumination optical path remain as for FIGS. 3A-3B, including elements 20, 2, 8A, 6, 8B and 4 of the cylindrical correction unit 214, except that light source 20 does not provide an objective test illumination source. Relay lenses 400, 500 and folding mirror 502 are also shown again.

Scale bar 550 indicates approximate sizes of the optical arrangements of FIG. 4. As an example, arrangements in other embodiments may be understood, in some embodiments, to be scaled similarly; e.g. with the beam envelope in telecentric regions of beam paths being about 2 cm wide. These measurements are non-limiting. It is readily understood to a person of ordinary skill in the art that particular lens powers, folding mirror arrangements, telecentric zone lengths, focal lengths, and other parameters of the system are optionally varied in suitable relationships to maintain the overall functional relationships of optical elements to each other, and functionality of the optical system overall, e.g. as descried herein. Examples of focal lengths of lenses, for example, may be estimated from pupils and/or focus points illustrated in the various drawings.

Objective testing is performed through an optical pathway wherein a beam from objective testing illumination source 200 impinges on beam splitter 301, is directed toward the eye through beam combiner elements 408A, 408B (corresponding to beam combiner 408 of other embodiments), and then to eye pupil 412 and retina 110 of eye 108. Returning light passes through beam splitter 301 and relay lenses 402, 401 to reach detection unit 102.

It should be noted that the arrangement of beam combiner elements 408A, 408B leaves open an unobstructed light path leading straight from eye 108. In some embodiments, beam combiner element 408B is provided as a partially reflective mirror, allowing light from straight beyond it to be combined as well. In some embodiments, a beam is brought into beam combiner element 408B configured to produce a focused image on the retina of the scene beyond the optics, e.g., using a suitable arrangement of one or more prisms, optionally comprising lenses and/or mirrors. A potential advantage of this is that the subject can be asked to look into the distance of their actual surroundings to encourage lens accommodation, and or eye positioning. Relative illumination from the scene and/or device-internal illumination (e.g., the subjective test target beam) can be adjusted as appropriate to provide suitable visual contrast to allow simultaneous viewing of each. Optionally, test targets are presented in such a way (e.g., with suitable focus and optionally with a suitable binocular eye vergence so as to produce a binocularly registered image) that the subject perceives a testing target as present in the scene at a particular distance. Optionally—for example, when testing a child or uncommunicative subject—the tester can use the surrounding pass-through to present any available and attention-attracting physical object at any distance available within the testing space to assist the testing process.

In some embodiments, additionally or alternatively, passthrough through combiner element 408B is used to incorporate a light beam originating from an additional display device: for example an integrated display screen, and/or a larger display screen set up in the surroundings. This has potential advantages, e.g., to allow a remotely-supervised testing session, in which the subject wearing the device is able to see and optionally interact with a testing supervisor (examiner) who is telecommunicating through the screen. The testing supervisor optionally gives all needed assistance to the subject through the telecommunication link. Optionally, there is an on-site assistant (technician) who assists in some portions of arrangements. Optionally, the testing supervisor supervises more than one test at a time, e.g. giving group and/or individual instructions during a telecommunication session involving a plurality of subjects and testing devices, wherein both one-to-one and group interaction is enabled, according to the needs of the moment and/or the particulars of the local telecommunication equipment configuration. The test presented may comprise objective and/or subjective visual acuity testing, in any suitable order of test presentation, and optionally with subjective and objective testing occurring at least in part simultaneously.

With adjustments to their optics as appropriate, a surroundings and/or auxiliary display pass-through arrangement is optionally provided together with any of the other embodiments presented herein.

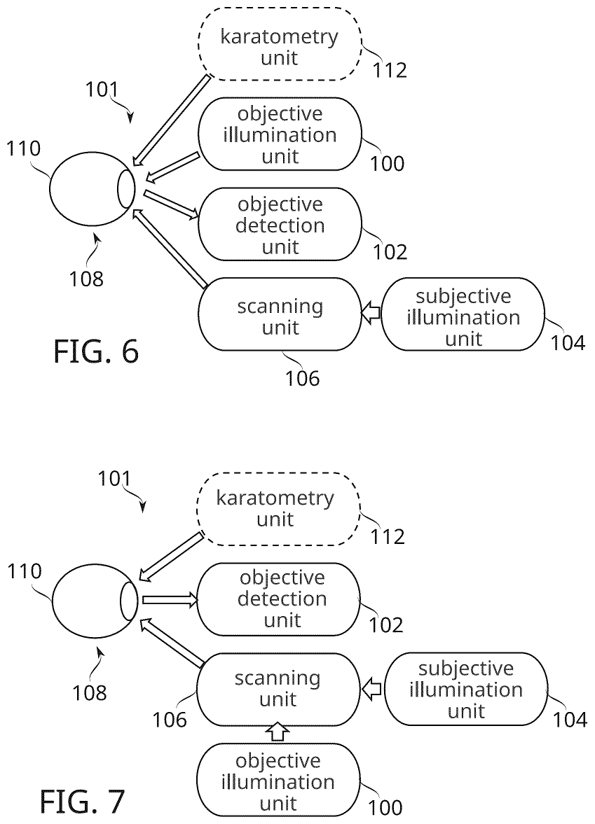

Additional details regarding functionality for various components and/or subsystems of FIGS. 3A-4 are described in relation to remaining figures herein, including embodiments described in relation to FIGS. 6-20C.

### Combined Cylindrical and Spherical Correction

Reference is now made to FIG. 5A, which schematically illustrates a cylindrical refractive error correction unit 214 in combination with a spherical refractive error correction unit 218, according to some embodiments of the present disclosure.

Details of the cylindrical refractive error correction unit 214 include lenses 2, 4, 6, 8 configured, in this example, as described in relation to FIG. 1A; however, this example should not be considered limiting.

Also shown are relay lenses 400, 500; and eye 108 comprising retina 110 and anatomical pupil 412.

Within spherical correction unit 218 are shown two fixed mirrors 522, 523 within the main beam path, and a pair of moving mirrors 521, which reflect light diverted from the initial direction of the beam path at mirror 522 back onto mirror 523, from where the light continues on to relay lens 500.

Moving mirrors 521 move toward or away from mirrors 522, 523, lengthening or shortening the overall beam path. This in turn adjusts spherical aberration of individual beams, e.g, it changes whether they come to a focus at, before, or after the position of retina 110. Since the beams are collimated to each other, the overall beam envelope does not change as a result of adjustments to the position of moving mirrors 521.

### Screen Illumination Sources

Reference is now made to FIG. 5B, which schematically illustrates a cylindrical refractive error correction unit 214 in combination with a screen illumination source 20A, according to some embodiments of the present disclosure. In some embodiments, the screen is a display panel; for example using μLED,kl μOLED, OLED, QDLED, LCD or another display technology.

The functions of lenses 4, 6, and 8, in this example, remain as described in relation to other embodiments providing cylindrical aberration correction, with pupil 14 being formed beyond relay lens 4, and conjugate to pupil 412 formed at eye 108 after passing through the relay formed by relay lenses 400, 500. Also shown is retina 110, on which an image of screen 20A is formed during optical acuity testing.

Since screen illumination source 20A is initially extended spatially, it may be considered as performing the roles of both light source 20 and relay lens 2 as described in relation to FIG. 1A. The spread of the light from each pixel of screen illumination source 20A may be angularly wider than shown; just the envelope of light paths that pass through pupil 14 is shown. Adjusting the position of screen illumination source 20A along the optical axis acts to adjust spherical corrective refractive power, e.g., performing the function of the movement of moving mirrors 521 in FIG. 5A.

### Eye Refraction Testing Systems

Reference is now made to FIG. 6, which schematically illustrates a block diagram of an optical system 101 combining objective and subjective eye refraction testing subsystems, according to some embodiments of the present disclosure.

In some embodiments, subjective testing and objective testing optical subsystems of optical system 101 share combined use of a retinal scanning display used to produce test images. In some embodiments, optical system 101 includes objective illumination unit 100 (which produces illumination reflected and measured for determining objective test results), an objective test detection unit 102 (which performs detection of the reflected objective illumination light), a subjective illumination unit 104, and a scanning unit 106.

Optionally, scanning unit 106 comprises a scanning mirror such as a MEMS (micro electromechanical system) mirror, galvo mirror or other reflective or transparent element used to produce beam scanning. For use in objective measurement, the objective illumination unit 100 illuminates the subject's eye 108. Light reflected by a layer in the subject's eye 108 returns in part to the objective test detection unit 102. The reflected light reaching the objective test detection unit 102 contains refractive information from the subject's eye 108. For subjective measurement, the subjective illumination unit 104 emits one or more beams (e.g., beams of different colors) which are two-dimensionally scanned (i.e., deflected to different angles which change rapidly in time so as to produce the impression of a single image) by scanning unit 106. The scanned light enters the subject's eye 108 and creates an image on the subject's retina 110. Optionally, the subjective illumination unit 104 and scanning unit 106 are used to form images (focused or unfocused) used to fixate the gaze and/or control lens accommodation (e.g., accommodation relief) of the subject eye 108 during objective testing.

Optionally, optical system 101 includes a keratometry unit 112. Keratometry unit 112 is configured to measure corneal shape of the subject's eye(s). This allows acquiring further and/or confirming information regarding optical functioning of the subject's eye 108; for example: regarding astigmatism, astigmatism axis and/or total corneal power, and/or the cornea's radius of curvature.

Reference is now made to FIG. 7, which schematically illustrates a block diagram of a variant of optical system 101 combining objective and subjective eye refraction testing subsystems, and having a dual-use scanning unit 106, according to some embodiments of the present disclosure.

The optical system 101 of FIG. 7 also includes objective illumination unit 100, objective test detection unit 102, subjective illumination unit 104 and a scanning unit 106. In this example, one or more beams emitted from the objective illumination unit 100 are directed by scanning unit 106 to the subject's eye 108, with the location of impingement and/or angle of incidence on eye 108 being affected by the position of scanning unit 106.

A portion of the light is then reflected by a layer in the subject's eye 108 to the objective test detection unit 102. The reflected light reaching the objective test detection unit 102 contains refractive information from the subject's eye 108.

As before, the subjective illumination unit 104 emits one or more beams which are two-dimensionally scanned, also by scanning unit 106. The scanned light enters the subject's eye 108 and creates an image on the subject's retina 110. Optionally, the subjective illumination unit 104 and scanning unit 106 are used to form images (focused or unfocused) used to fixate the gaze and/or control lens accommodation (e.g., accommodation relief) of the subject eye 108 during objective testing. Light from the two sources may be presented simultaneously (e.g., where light from objective illumination unit 100 and subjective illumination unit 104 may be separable by a property such as wavelength and/or polarization to avoid interference with the function of objective test detection unit 102), or optionally presented in rapid alternation.

Optionally, optical system 101 includes a keratometry unit 112. Keratometry unit 112 is configured to measure corneal shape of the subject's eye(s). This allows acquiring further and/or confirming information regarding optical functioning of the subject's eye 108; for example: regarding astigmatism, astigmatism axis and/or total corneal power, and/or the cornea's radius of curvature.

### Optical Modules of Eye Refraction Testing Systems

Reference is now made to FIG. 8, which schematically illustrates a block diagram illustrating optical modules of a variant of optical system 101 combining objective and subjective eye refraction testing subsystems, and having a dual-use scanning unit 106, according to some embodiments of the present disclosure. In some embodiments, optical system 101 of FIG. 8 corresponds to a more modularly detailed illustration of optical system 101 of FIG. 7. In particular, indication of certain optionally common units is shown, along with certain modular details of correction and relay optics.

In some embodiments, objective illumination unit 100 itself comprises an objective beam source 200 and associated collimating and focusing optics 202.

The beam source 200 comprises, for example, one or more light emitting diodes and/or laser diodes such as edge emitters or VCSELs in the IR range (>640 nm). Optionally, beam source 200 produces light in another wavelength range, e.g., in the visible or UV range.

Collimating and focusing optics 202 comprise, for example, one or more lenses (spherical and/or cylindrical), configured to focus and/or collimate the beam emitted from beam source 200. Collimating and/or focusing optics 202 optionally include a constant or variable-diameter pin hole.

Scanning unit 106 and collimating optics 204, in some embodiments, form a functional group of modules shaping and transmitting the beam from objective illumination unit 100 toward the eye. Collimating optics 204 comprises, for example, one or more lenses, of which one or more is moveable along the optical axis of this beam. Additionally or alternatively, collimating optics 204 comprises one or more tunable lenses; for example, lenses that change focus by electrically controlling the curvature of a meniscus between two immiscible liquids, and/or change focus by another means.

From collimating optics 204, the beam from objective illumination unit 100 transmits to optics-to-eye 206, optically configured to direct the beam to the subject's eye 108. For example, optics-to-eye 206 comprises one or more lenses and/or folding mirrors. Optionally, optics-to-eye 206 includes one or more optical elements which combine or separate beams. In some embodiments, these include an optical element (e.g., a dichroic beam splitter) that allows light to pass through or be reflected from it depending on the light's wavelength. For example, it is optionally configured to let IR light pass, while reflecting visible light. Optionally, it is configured to reflect a portion of the IR light, but let another portion pass, e.g., so that that light can pass through it to the eye, but then (upon returning from the eye), be diverted at least in part (and preferably mostly, e.g., at least 50%, 60%, 70%, 80%, 90% of returning light) to a detector. Additionally or alternatively, there is provided an optical element having refraction properties sensitive to polarization. For example, light polarized in s-polarization refracts from this element differently than light polarized in p-polarization. In some embodiments, there is provided a diffractive optical element such as a diffractive beam combiner or splitter, or an optical wave guide. The splitting/combining elements are arranged to suitably direct light from the different sources of optical system 101 to eye 108, and/or direct light reflected from eye 108 to an appropriate detector, e.g., objective test detection unit 102.

Objective test detection unit 102 is configured to detect reflection from the subject's eye 108; and in particular, light received by the subject's eye 108 from objective illumination unit 100. In some embodiments, the detector of objective test detection unit 102 comprises one or more photo diodes, a CCD camera, a PSD (Position Sensitive Detector) and/or another photosensor. Optionally, objective test detection unit 102 comprises optics configured to compensate (at least approximately) for the refractive power of the subject's eye 108, and to focus the image of the retina 110 onto the detector. Examples include: a moveable and/or tunable lens positioned before the detector, detector and lens configured as a movable unit relative to a lens placed between them and the subject's eye 108, and a moveable or tunable lens placed between the subject's eye 108 and the lens and detector unit.

In some embodiments, the subjective testing optical subsystem defines an optical pathway including a subjective illumination unit 104 comprising a subjective testing beam source 210 and combining and collimating optics 212. Beam source 210 comprises, for example, one or more laser diodes (e.g., edge emitters or VCSELs), emitting in the visible range (440-660). Preferably the beam source 210 includes at least three beam sources: a red beam source (e.g., wavelength approximately between 620 nm to 660 nm), a green beam source (e.g., wavelength approximately between 500 nm to 540 nm), and a blue beam source (e.g., wavelength approximately between 440 nm to 470 nm). combining and collimating optics 212 comprise, for example, one or more optical elements to combine beams. For example, combining and collimating optics 212 comprises one or more of:
An optical element that allows light to pass or be reflected depending on the light's wavelength.
A diffractive optical element such as a diffractive beam combiner.
An optical wave guide, for example, on a photonic integrated circuit.

Combining of the beams is optionally before or after being collimated by one or more collimating lenses. Where beams of multiple wavelengths are to travel together, they are preferably combined so as to match their vergences. Combining and collimating optics 212 optionally includes a pin hole.

In some embodiments, cylindrical correction unit 214 (wherever it is placed) corresponds to an arrangement of lenses operable to introduce a selected axis and power of cylindrical optical correction to the beam subjective illumination unit 104; for example, as described in relation to FIGS. 1A-2B.

Alternatively, in some embodiments, there are provided one or more chambers, providing among them for a plurality of cylindrical lens powers to be introduced into the beam. Optionally, each of a plurality of chambers is selectable by rotation (or exchanging motion), to introduce a different cylindrical lens power. Additionally or alternatively, one or more chambers is provided with Jackson's cross cylinder lenses, adjustable relative to each other to introduce a range of different cylindrical lens powers. Selection and/or adjustment sets the current optical power compensating for the subject's cylindrical and axis error.

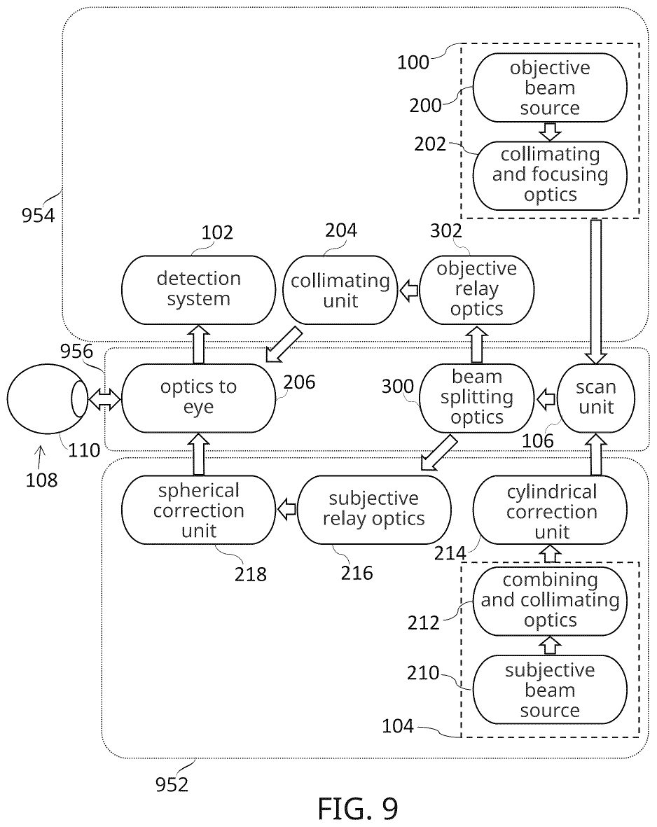

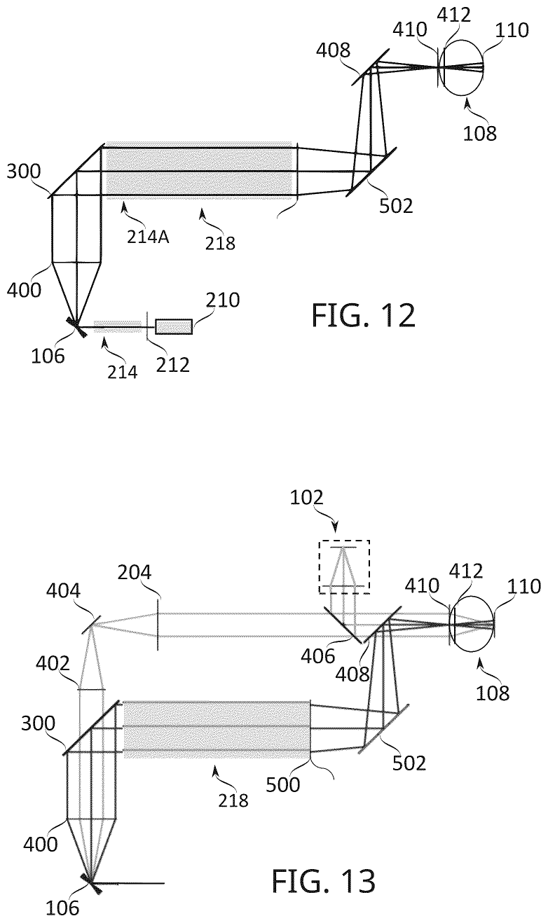

The location of cylindrical correction unit 214 within the optical path is not necessarily in the same order as shown with respect to scan unit 106, subjective relay optics 216 and/or spherical correction unit 218. For example, it may generally be rearranged in a position also as described with respect to spherical correction unit 218, before or after it. It is a potential advantage in particular to place cylindrical correction unit 214 after scan unit 106, as this avoids complicating the cylindrical correction with wavefront effects caused by the varying angles of reflection from the mirror. This applies also to correction unit 214 as shown and discussed in relation to other figures, for example, FIGS. 9 and 12.

In some embodiments, scanning unit 106 is shared between the subjective testing and objective testing optical subsystems of optical system 101.

Subjective relay optics 216 comprises, for example, one or more lenses that relay the beam from scanning unit 106 to a chosen plane; for example, to the subject's pupil, with a chosen magnification. Alternatively, the beam is relayed with a chosen magnification to an intermediate plane before further relay to the subject's pupil.

Spherical correction unit 218, in some embodiments, comprises, for example, a moveable and/or tunable lens that moves along the optical axis of the laser beam from subjective illumination unit 104. It is shown placed in the optical path after subjective relay optics 216. Alternatively, it may be positioned between the beam source 210 and the scanning unit 106 (e.g., as for cylindrical correction unit 214). In another example, spherical correction unit 218 optionally comprises one or more movable mirrors and/or lenses placed between the lenses of the subjective relay optics 216.

In the position shown, spherical correction unit 218 includes optics which direct light to optics-to-eye 206. Within optics-to-eye 206, some elements are optionally shared in common with optical path(s) of the objective testing optical subsystem. For example, a beam combiner and/or folding mirrors may be shared. Other elements may have functions used by only one of the testing functions, for example, folding mirrors and/or lenses.

In some embodiments, along light paths(s) of the objective testing optical subsystem: a beam emitted from the objective beam source 200 is collimated and/or focused by collimating and focusing optics 202. This beam is then reflected by scanning unit 106 to collimating optics 204, which together with scan unit 106, control, for example, the beam's location and/or angle of impingement onto the subject's eye 108 after passing through optics to the eye 206. Light reflected from a layer in the subject's eye returns to optics-to-eye 206, and is then reflected to objective test detection unit 102. The reflected light reaching the objective test detection unit 102 contains refractive information from the subject's eye 108, which is subjected to further analysis, e.g., according to methods of objective refraction measurement known in the art.

In some embodiments, along light paths(s) of the subjective testing optical subsystem: one or more beams emitted from the subjective beam source 210 are collimated/combined by combining and collimating optics 212. The one or more beams pass through cylindrical correction unit 214 and are two-dimensionally scanned by scanning unit 106. Cylindrical correction unit 214 is not necessarily at this location; e.g., optionally it is positioned just before or after spherical correction unit 218, or at another location after scanning unit 106. Subjective relay optics 216 relay the scanned beams to a chosen plane; for example, the subject's pupil. The scanned beams pass through the spherical correction unit 218 and optics-to-eye 206. The scanned light enters the subject's eye 108 and creates an image on the subject's retina 110. During subjective visual acuity testing, the visual appearance of the image is reported by the subject. Settings of spherical correction unit 218 and/or cylindrical correction unit 214 are adjusted accordingly to determine settings that produce the reported best image according to the reported perceptions of the subject.

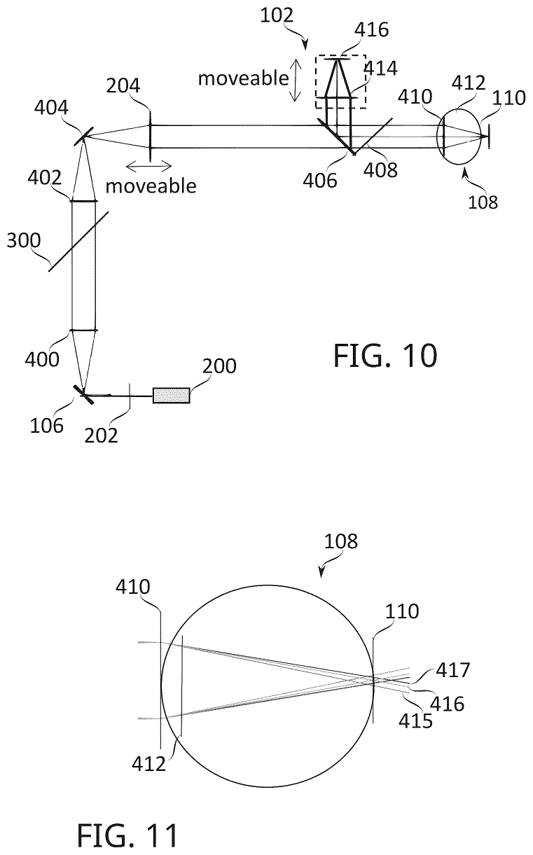

Reference is now made to FIG. 9, which schematically illustrates a block diagram illustrating optical modules of a variant of optical system 101 combining objective and subjective eye refraction testing subsystems, having a dual-use scanning unit 106, according to some embodiments of the present disclosure. In some embodiments, optical system 101 of FIG. 9 corresponds to a variation of optical system 101 of FIGS. 7 and 8. In particular (compared to FIG. 8), indication of objective relay optics 302 and beam splitting optics 300 is shown. The modules added in FIG. 9 compared to FIG. 8 assist support for objective refractive error measurement based on the Scheiner principle (e.g., as described in relation to FIGS. 10-11). Also shown are boxed indications identifying modules of the objective testing optical subsystem (box 954), the subjective testing optical subsystem (box 952), and modules common to each (box 956).

In some embodiments, the objective testing optical subsystem of optical system 101 comprises an objective illumination unit 100, including beam source 200 and collimating and focusing optics 202. Also included is scanning unit 106. Beam splitting optics 300 comprise one or more optical elements configured to separate beams; for example, an optical element that allows light to pass or be reflected depending on the light's wavelength. In some embodiments, such an optical element is configured to let IR light pass, while reflecting shorter-wavelength visible light. In some embodiments, the optical element has refraction properties sensitive to polarization. For example, it refracts s-polarized light differently than p-polarized light. In some embodiments, the beam splitting optics comprise a diffractive optical element; for example, a diffractive beam splitter.

Objective relay optics 302 comprise, for example, two or more lenses which relay the beam from scanning unit 106 to a chosen plane such as the subject's cornea, retina 110, or other chosen plane in the eye; and with a chosen magnification. Also provided are collimating optics 204, optics-to-eye 206, and objective test detection unit 102, configured to detect reflection from the subject's eye 108.

In some embodiments, the subjective testing optical subsystem of optical system 101 comprises a subjective illumination unit 104, including beam source 210 and combining/collimating optics 212. Also provided, in some embodiments, are cylindrical correction unit 214 (again, not necessarily at this location; e.g., optionally just before or after spherical correction unit 218 or at another position after scanning unit 106), scanning unit 106 (shared in common with the objective testing optical subsystem), beam splitting optics 300 (also shared in common), subjective relay optics 216, spherical correction unit 218 and optics-to-eye 206 (again, shared in common).

Some elements of modules shared in common may be individually assigned to just one of the two testing subsystems. For example, there may be a common beam combiner, but one or more lenses, folding mirrors or other elements separate.

Reference is now made to FIG. 10, which is a schematic optical diagram of the objective testing optical subsystem of optical system 101 of FIG. 9, according to some embodiments of the present disclosure.

In some embodiments, beam source 200 emits a beam which focusing optics 202 changes the diameter of, approximately focuses onto a scanning unit 106. Scanning unit 106 reflects the beam to optional folding mirror 404 through lenses 400 and 402, which in FIG. 10 implement the objective relay optics 302 of FIG. 9. The beam also passes through beam splitting optics 300.

The beam continues to collimating optics 204, optionally implemented as a moveable lens to allow use of the Scheiner principle discussed in relation to FIG. 11. The focus length of collimating optics 204 approximately matches the distance to folding mirror 404. More generally, the focus length approximately matches the distance to the focus point of objective relay 302. This allows beams that were reflected at different angles from scanning unit 106 to return to propagating parallel to each other, although positioned differently in space.

The beam continues next through optics-to-eye 206, illustrated comprising beam splitter 406 and beam combiner 408. The beam enters the subject's eye 108 through cornea 410 and pupil 412. Some light reaching retina 110 is reflected back from the retina 110 to the pupil 412 and the cornea 410.

The reflected beam continues passing through beam combiner 408 and reaches beam splitter 406. Beam splitter 406 reflects the light to the objective test detection unit 102, illustrated as comprising lens 414 and detector 416. The reflected light from the retina 110 reaching the objective test detection unit 102 contains refractive information from the subject's eye 108, which may be analyzed according to the Scheiner principle, e.g., as next explained.

Reference is now made to FIG. 11, which is a schematic optical diagram of the Scheiner principle, performed, for example, using the optical arrangements of FIG. 10, according to some embodiments of the present disclosure.

Three pairs of laser beams 415-417 are shown entering the subject's eye 108 from the left. The beams in each pair are parallel to each other, but each pair of beams enters the subject's eye 108 at a (slightly) different angle. In operation, each pair represents a different position of, e.g., lens 204 as it moves to change the vergence of the beams. There are optionally also a plurality of positions (for example, at least three) at which beams enter the eye (e.g., at different meridians of the eye), and those beams may be presented simultaneously with each other.

Near the retina, the location of the crossing of each beam pair depends on the incident angle of the beam pairs, as modified by the magnifying power of the anterior segment of the eye (e.g., lens and cornea) to pass through pupil 412.

By controlling beam source 102 and scan unit 106 in correlation with adjustments to collimating optics 204, it is possible to control both the incident angle of the beam pairs and their location when impinging on the subject's eye 108. Suitable adjustment results in each pair's crossing exactly on the retina 110. This state is detected by imaging the retina onto the detector 416, which can be coordinated to move together with lens 414 when another lens is between lens 414 and the eye; or lens 414 can move relative to detector 416 to compensate for the refractive error of the subject's eye 108. The position of collimating optics 204 and scan unit 106 when the subject's eye 108 is focally illuminated by beam source 102 corresponds to a certain refractive error in the specific locations of the subject's eye 108 on which the beam impinged. With suitable selection of a plurality of locations on the anterior segment of the subject's eye 108 to receive beams, values of spherical cylindrical and axis of the subject's eye 108 can be derived.

During the objective measurement, the subject is encouraged to focus into the distance, maintaining a relatively weak accommodation. The subject is instructed, for example, to attend to a target (e.g., a blurred image) which is experienced as perceptually distant encouraging them to accommodate lenses of their eye(s) accordingly. In some embodiments, the target is created by a retinal scanning display system used also as the subjective testing optical subsystem for subjective visual acuity examination.

Reference is now made to FIG. 12, which is a schematic optical diagram of a subjective testing optical subsystem, according to some embodiments of the present disclosure. In some embodiments, the arrangement of FIG. 12 corresponds to the elements of blocks 952 and 956 of FIG. 9.

Beam source 210 emits one or more beams that go through combining and collimating optics 212 and cylindrical correction unit 214; placed, for example, in the corresponding gray area. Optionally, cylindrical correction unit 214 (corresponding in construction to cylindrical correction unit 214) is placed elsewhere in the optical path, for example, just before or just after spherical correction unit 218. In some embodiments, this corresponds to any of the cylindrical and axis correction arrangements described in relation to FIGS. 1A-2B.

In some embodiments, the one or more beams are reflected from scanning unit 106 through relay lens 400 to beam splitter 300. These elements are optionally used in common with the objective testing optical subsystem, e.g., of FIG. 10. At beam splitting optics 300, objective and subjective illumination beams are split (e.g., the subjective illumination is reflected, while in FIG. 10, the objective illumination passes through).

The one or more beams pass through the spherical correction unit 218 (placed, for example, in the corresponding gray area), and through another relay lens 500.

As shown, subjective relay optics 216 comprises relay lenses 400 and 500 which relay the one or more beams onto the subject's pupil 412.

After relay lens 500, the one or more beams are directed to the subject's eye 108 by optics-to-eye 206. As shown, optics-to-eye 206 comprise folding mirror 502 and beam combiner 408 (shown also in FIG. 10). The one or more beams continue passing through the cornea 410 and pupil 412 to reach the retina 110 where they create an image.

Reference is now made to FIG. 13, which is a schematic optical diagram of combined objective and subjective testing optical subsystems of an optical system 101, according to some embodiments of the present disclosure. In some embodiments, the arrangement of FIG. 13 corresponds to many of elements of FIGS. 10 and 12, in combination. The objective and subjective illumination beams are shown as having been previously combined together (by further elements not shown) before being reflected by scanning unit 106 at the same angle.

Beam-combining optical elements include, for example: an element that allows light to pass or be reflected depending on the light's wavelength; a diffractive optical element such as a diffractive beam combiner; and/or an optical wave guide, for example, on a photonic integrated circuit. Combining of beams may occur before or after being collimated by one or more collimating lenses. Alternatively, the beams impinge on the scanning unit 106 at different angles, and spatial relationships of components further along the remainder of the optical path are suitably adjusted for the difference.

As shown, the reflected beams from scanning unit 106 for the objective and subjective measurements reach lens 400 and are separated by beam splitting optics 300. The beams for the objective measurements pass through objective relay optics 302, comprising lens 402 and lens 400. The beam is then reflected by folding mirror 404 through collimating optics 204, passes beam splitter 406, and passes beam combiner 408. Beam combiner 408 directs the beams used for objective and subjective measurements to the subject's eye 108. Reflection from the subject's eye 108 passes through beam combiner 408 and is reflected by beam splitter 406 into detection unit 102, where the reflection light is analyzed.

Along the subjective illumination optical path, relay lens 400 serves as the proximal relay lens of subjective relay optics 216. Beam(s) are separated from the objective illumination beam at beam splitting optics 300 (e.g., reflected). The beam(s) pass through spherical correction unit 218 (placed, for example, in the corresponding gray area) to reach the distal relay lens 500 of subjective relay optics 216, from which the beam(s) are relayed onto the subject's pupil 412. Relay to eye 108 is via optics-to-eye 206, illustrated as comprising folding mirror 502 and beam combiner 408. The beam(s) enter the subject's eye 108 and reach the retina 110 where they create an image.

Optionally, optical system 101 of FIGS. 9-10 and 12-13 includes a keratometry unit 112. Keratometry unit 112 is configured to measure corneal shape of the subject's eye(s). This allows acquiring further and/or confirming information regarding optical functioning of the subject's eye 108; for example: regarding astigmatism, astigmatism axis and/or total corneal power, and/or the cornea's radius of curvature.

### Dual-Scanning Unit Arrangement

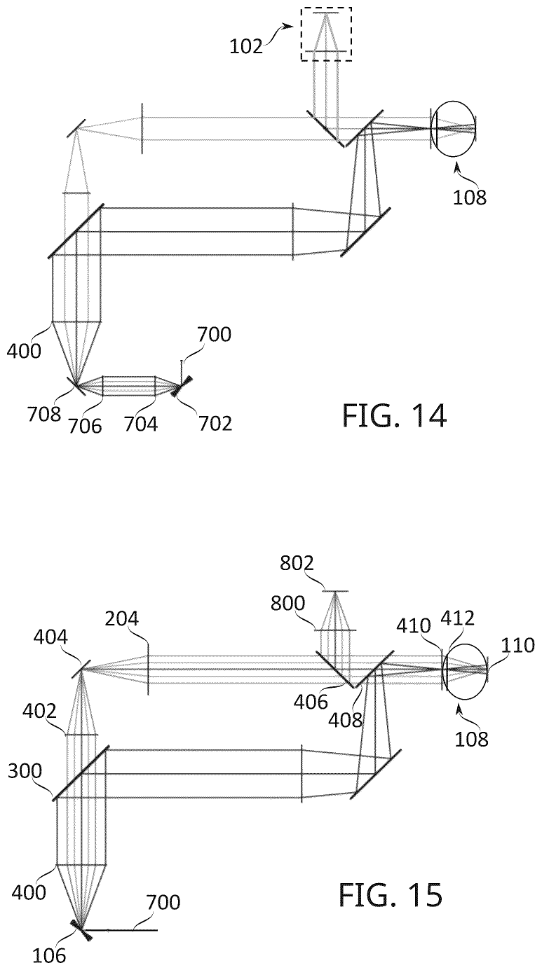

Reference is now made to FIG. 14, which is a schematic optical diagram of combined objective and subjective testing optical subsystems of an optical system 101 using two scanning units 702, 708, according to some embodiments of the present disclosure. Each of the scanning units 702, 708, scans in one dimension. They are arranged so that together they can perform a two-dimensional scan.

Combined beams 700 of the objective and subjective optical subsystems impinge on the first scanning unit 702 and are relayed via lenses 704, 706 to a second scanning unit 708. Combining of the beams is not shown. The beams continue to lens 400 and from there to the subject's eye 108 through arrangements of elements which are, for example, corresponding to arrangements of FIGS. 9-10 and/or 12-13.

The relay between the two scanning units 702 and 708 indicated by two lenses, is optionally implemented by any suitable number of lenses. Optionally, there are no relay components positioned between scanning units 702 and 708.

Objective Optical Testing with Ray Tracing Aberrometry

Reference is now made to FIG. 15, which is a schematic optical diagram of combined objective and subjective testing optical subsystems of an optical system 101, with the objective testing optical subsystem using ray tracing aberrometry, according to some embodiments of the present disclosure. The objective and subjective illumination beams are shown as having been previously combined together (by further elements not shown) before being reflected by scanning unit 106 at chose angles for each exam.

Regarding beam(s) used in objective testing measurements, combined beams 700 impinge on scanning unit 106, and may be internally diverging, converging, or collimated as they do so. For example, they may be focused or approximately focused when impinging on scanning unit 106.

Beam-combining optical elements include, for example: an element that allows light to pass or be reflected depending on the light's wavelength; a diffractive optical element such as a diffractive beam combiner; and/or an optical wave guide, for example, on a photonic integrated circuit. Combining of beams may occur before or after being collimated by one or more collimating lenses.

Scanning unit 106 reflects the beams to beam splitting optics 300, which separate the objective test illumination beam(s) from those of the subjective and/or objective focus target illumination pathway.

Beam(s) for objective measurements pass objective relay optics 302 (FIG. 9); as shown in FIG. 15 comprising lens 402 and lens 400. In some embodiments, the beams reflect from folding mirror 404. Collimating optics 204, illustrated as a stationary lens, optionally comprises one or more stationary, moveable, and/or tunable lenses.

Beams reflected at different angles from the scanning unit 106 propagate parallel to each other while collimated, though positioned differently in space. The beams continue through beam splitter 406 and beam combiner 408 and enter the subject's eye 108, passing through the cornea 410 and pupil 412. The beams reach the retina 110 and are reflected back from the retina 110 to the pupil 412 and the cornea 410. The reflected light continues passing through beam combiner 408 and is reflected by beam splitter 406 to the lens 800 and onto a detector 802.

Beam splitter 406, beam combiner 408, lens 800 and detector 802 are optionally fixed relative to each other; but moveable together and/or provided together with another lens before them that is moveable to compensate for refractive error. Detector 802 is implemented, for example, using a photo-sensor, a quad photo-detector, a CCD camera, a positioning sensitive detector (PSD), and/or another photosensor.

The reflected light from the retina 110 reaching detector 802 contains refractive information from the subject's eye 108. Different beams resulting from different angles of scanning unit 106 are illustrated. All the beams are parallel to each other upon reaching the subject's eye 108, but enter the subject's eye 108 at different locations. By controlling beam source 200 (contributing to combined beam 700) and scan unit 106 it is possible to sequentially change the location at which the beam impinges on the subject's eye 108. The detector 802 measures the exact location where each beam reaches the retina 110 by means of the retro-reflected light. This process continues until several separated points are projected into the entrance pupil 412. This way a correlation is obtained between the direction that the light beams have taken while entering and leaving, allowing a reconstruction of the real wave-front error. From such data, values of spherical, cylindrical, axis and corneal curvature of the subject's eye 108 can be derived. The subjective visual acuity exam subsystem is optionally configured, for example, as described in relation to embodiments of FIGS. 12-14.

### Dual-Scanning Unit Arrangement

In some embodiments, the general arrangements of FIG. 15 are modified by combination with the dual-scanning unit arrangement described in relation to FIG. 14. Again, each of scanning unit scans in one dimension; together they can perform a two-dimensional scan.

Testing of Visual Acuity and/or Refractive Error

For use in subjective visual acuity testing, the image formed by the scanned beam on the retina 110 comprises, for example, a visual acuity exam chart such as a Snellen chart, Landolt ring chart, tumbling E chart, Lea test, HOTV chart, clock dial chart, sunburst chart, spatial frequency chart (e.g., EIA Resolution Chart 1956, ISO 12233, USAF 1951) and/or any other image or images for evaluating the subject's refractive error. Optionally, 3-D and/or binocular presentation capabilities used to present visual test images as apparently three dimensional and/or localized in depth. For example, depth cues may be used to modify the apparent size/distance of stimuli, optionally while retaining the same angular size. This may assist in controlling accommodation, for example. Optionally, stimuli associated with different depths (that is, different depth cues) are presented simultaneously, resulting in a three dimensional appearance of the test image.

As a starting point for the subjective exam, different information can be used for shaping the beam forming in the initial images. For example, the subject's last prescription, or the subject's glasses can be measured or the values obtained previously in the objective visual acuity exam is used. Initial values for spherical, cylindrical and/or axis are used to shape the scanned laser beam which produces the image. Values used for the starting point can be used as initially obtained or with an added or subtracted factor (e.g., a calibration factor, which may be determined empirically).

Based on input from the subject, the spherical, cylindrical and axis are adjusted, to seek parameters producing an image on the subject's retina 110. reported by the subject as optimal.

The changes to the beam's shape (divergence/convergence, astigmatism and astigmatism axis, corresponding to spherical power, cylindrical power, and cylindrical axis) to produce the optimized image on the subject's retina 110, are used to derive the subject's subjective values for spherical, cylindrical and axis refractive errors. Following the subjective visual acuity exam, a complete eyeglass prescription can be produced.

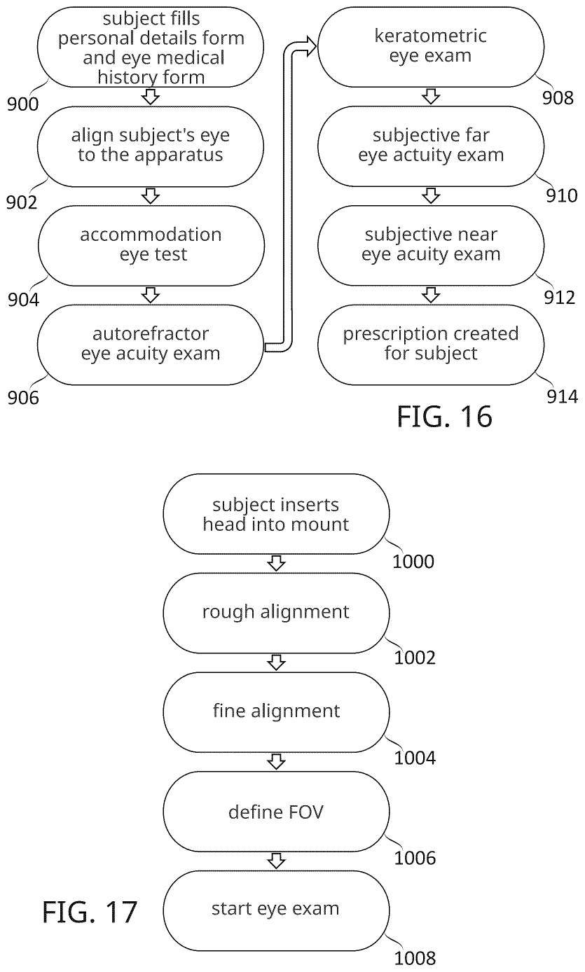

Reference is now made to FIG. 16, which is a schematic flowchart of a method for measuring the refractive error of subject's eyes and providing a prescription for eyeglasses or contact lenses, according to some embodiments of the present disclosure. The examiner, the subject themself, or a designated person performs and/or supervises the operations of each block, optionally remotely. In some embodiments a computer processor is used to operate aspects of device function, e.g., in response to sensed information, in response to remotely and/or locally input commands, and/or following one or more internally defined protocols.

At block 900, in some embodiments, the subject is optionally asked to fill in (or otherwise provide) personal information and relevant known medical eye history. At block 902, in some embodiments, the subject's eye is aligned correctly to the apparatus; for example, as detailed in relation to FIG. 17.

Brief reference is now made to FIG. 17, which is a schematic flowchart of a method to align a subject's eye to an optical system 101, according to some embodiments of the present disclosure.

At block 1000, in some embodiments, the subject inserts their head onto a mount. This comprises, in some embodiments, a chin rest, forehead rest, and/or cheek bone rest; and/or another mechanical arrangement to keep the subject steady in a certain place.

At block 1002, in some embodiments, a rough alignment process is performed, continuing, for example, until cameras located in the apparatus are able to recognize and position the subject's pupils relative to the apparatus. This process can be performed manually (e.g., position adjustments are performed by hand) and/or automatically under control of the processor (e.g., adjustments are performed using actuators, under feedback control from a controller which is monitoring camera images or other sensor data monitoring mono-ocular and/or binocular eye alignment (e.g., pupil alignment) inside and/or outside the apparatus.

At block 1004, in some embodiments, e.g., after sensing recognizes the subject's pupil position, a fine alignment process is initiated (e.g., with further motor actuations, optionally under image or other sensor feedback-based control by the processor). This results in the pupil being in the correct position relative to the optical axis of the apparatus on, e.g., up to six axes (x, y, z, roll, pitch, and yaw). Optionally pitch and/or yaw adjustment is omitted. Optionally roll is omitted. Optionally one or two of the displacement axis is omitted or controlled manually.

At block 1006, in some embodiments, an optional confirmation of positioning is provided via images with marked boundaries being projected to the eye, with input from the subject indicating angles at which the projected beams enter the subject's pupil 412. These can be used to define the subject's field of view (FOV). Optionally, the FOV is determined automatically (e.g., by imaging of the retina). Optionally, the fine alignment process of block 1004 is relied on to ensure an appropriate FOV, or the FOV is otherwise assumed to be suitably arranged as a result of other arrangements.

From block 1008, in some embodiments, the eye exam proceeds, optionally for both eyes together, or the eyes in alternation.

Now with returning reference to FIG. 16, a first exam is initiated on the corrected positioned subject.

At block 904, in some embodiments, the subject's accommodation is optionally analyzed. The analysis may be based on initial attempts at objective testing, eye tracking, or another form of electronic sensing. The result potentially assists (e.g., is used by the processor) in determining device settings which can compensate for incomplete accommodation, such as the variation of spherical correction to the focus image. Optionally, extra measures are taken to promote accommodation, such as presentation of objects in a passthrough-visualized scene beyond the testing optics themselves, an image sequence presented (e.g., via the subjective illumination optical pathway) to draw the subject's attention, or another method. Initiation of such measures is optionally suggested by outputs of the processor to a user interface, and/or performed by the processor.

At block 906, in some embodiments, an objective refractive error exam is performed. Optionally operations of blocks 904 and 906 are performed together, e.g., iteratively as test results are obtained. The test is performed with the subject looking at a target (usually a blurred image, or an image including both blurred and sharply-presented elements), optionally created by the optical path used also (e.g., used later on and/or simultaneously) to perform a subjective visual acuity exam. In some embodiments, the objective refractive error exam is performed on both eyes simultaneously. In some embodiments, the testing device includes a moving element which actuates to adjust a relative angle between projection systems showing images to either eye during acuity testing. This adjustment may be used to change the apparent distance of a binocularly presented target. As the subject adjusts, there may be changes to eye vergence and lens accommodation, e.g., lens accommodation may be drawn to a distant focus. Due to the accompanying change in eye vergence, the angle of the subject's eye may also change. In some embodiments, adjusting the relative angle between projection systems also adjusts the angle of the optical path which projects objective testing light patterns onto the retina, thereby maintaining it in angular alignment with the eye. Adjustments are optionally performed under control of the processor as it follows a protocol, and/or upon sensed detection of the position and/or sensed state (e.g., pupil size, fixation stability, and/or accommodation) of the eyes of the subject.

In some embodiments, objective testing (e.g., infrared illumination-based objective testing) is performed at least partially simultaneously with other testing, e.g., the subjective testing measurements of blocks 910-912. Through simultaneous testing, there is potentially a savings of time, and/or an opportunity to enhance objective testing results, e.g., as a subject focuses to different distances during subjective testing. This may occur, for example, as a result of a clearer presentation of targets as the optical corrections used in subjective testing change. In some embodiments, the subjective test image itself is used as a target for the objective test; e.g., presented in depth (and optionally “swept” in depth, e.g., moved from apparent foreground to apparent distance during transitions in the testing) to help set an appropriate accommodation for ongoing objective testing. Such modulations of the subjective test image are optionally performed by the processor as part of a predetermined protocol, and/or in response to sensed state of the eyes of the subject.

At block 908, in some embodiments, a keratometry measurement is optionally performed, according to whether optics configured to perform this test are present (e.g., as described in relation to FIGS. 6 and 7, but optionally provided together within any of the optical system 101 embodiments described herein).

Beginning with block 910, in some embodiments, subjective measurements are optionally conducted, comprising one or both of a far visual acuity exam at block 910 (including examination for astigmatism), and a near visual acuity exam at block 912.

In some embodiments, tests make use of a binocular test target presentation capability to assist testing. In some embodiments, the subjective test comprises a switching of at least a portion of the presented target between two eyes, and/or presentation of different parts of the target to different eyes. The subject may be asked (optionally, prompted by the device itself) to evaluate, in effect, which eye sees the target more clearly, e.g., by being asked when they see the target most clearly, or what part of the target they see most clearly. In some embodiments, the response is provided directly to and/or measured by the device itself. In some embodiments, the processor follows a protocol, based on the inputs received, to determine how the optics of the device should be altered, e.g., to correct for spherical and/or cylindrical aberrations of the subject's eye(s). In some embodiments, targets presented to each eye are made sufficiently distinct (e.g., stripes of different orientations) that their separate adjustments can (potentially) be distinctly perceived and reported.

In some embodiments, testing optical power (cylinder and/or spherical) oscillates through a range with relative rapidity (e.g., slightly more quickly than the subject can respond before conditions have changed), and the subject is instructed to signal when they see a certain condition of clarity and/or matching. While there is potentially a lag in subject response, the oscillation may approach the signaled condition from opposite sides, so that the lag phase can be estimated (e.g., by the processor, for example by averaging response phases) to help determine the moment of the perceptual condition being signaled. This potentially helps to speed up the test process, and/or allows a larger and/or more heavily sampled number of optical correction conditions to be evaluated. Optionally, the rate of oscillation is adjusted to the response performance of the subject as appropriate, e.g., to maintain suitably consistent responses. Performed more slowly, the oscillation potentially helps determine if there is accommodation hysteresis (e.g., differences in eye accommodation depending on whether optical correction power is increasing or decreasing). Adaptation to patient performance is optionally performed automatically by the processor, based on patient performance characteristics such as consistency and/or hesitancy of responses received.

Perceptual simultaneity is not necessarily used. For example, cylinder power may be evaluated by movement of a cylinder lens arrangement at a relatively slow oscillation rate, e.g., 1 Hz or lower. A displayed pattern may be shown to the subject which advances (optionally smoothly) down, across, around, or in another manner through the display area. The subject may be instructed to look for (and optionally at) the region of greatest clarity. They may report it (e.g., name a number or other label positioned nearest that region, or otherwise describe it), and/or the eye position itself is optionally used as an indication of where optical clarity is perceived to be best.

In some embodiments, eye movements (e.g., as measured by an eye tracking detector inside the device) themselves lead the stimulus presentation and configuration of the optics. For example, cylinder power, cylinder axis, and/or spherical power may be mapped through positions on the display. As the subject looks over the display region, the optics are adjusted to match the mapping. The region where the subject's gaze is drawn and/or settles is, accordingly, selected as a basis for a further round of testing and selection, e.g., optionally after re-mapping. Re-mapping may be continuous, e.g., such that as the eye moves in a particular direction seeking a region where the target takes on greater focus, the mapping “scrolls” (perhaps more gradually) so that the eye is drawn back toward the center of the display area. Optionally, the optical task is varied during a test to reduce subject fatigue, e.g., switching between different ways of dividing the display area among optical settings, different target shapes and/or colors, and/or different patterns of target movement.

In some embodiments, intensity variations in the subjective testing illumination are used to help evaluate subject visual field capabilities, at least in some portion of the subject's visual field, e.g., near the fovea. For example, the subject may be asked to evaluate (and signal) when two illumination areas are matching and/or visibly distinct in intensity, when and/or where they can perceive a change in illumination intensity, when and/or where they can perceive an illumination gradient, or when/where another criterion is met.

In some embodiments, a subject is given at least partial control over the visual stimulus and/or optical correcting power, allowing them to manually select for themselves conditions at which a certain subjective perception criterion is met. Optionally, the selection is repeated from different initial conditions, and/or from among different available ranges. For example, the subject may rotate a knob (or control another selector) to select an orientation of cylindrical correction which leads to the greatest distortion and/or minimal distortion (e.g., clearest separation of two nearby lines or spots), and/or to select a power of cylindrical and/or spherical correction which leads to the least distortion, to distortion which matches among their two eyes, or according to another criterion.

At block 914, in some embodiments, a prescription for refractive glasses and/or contact lenses is optionally prescribed.

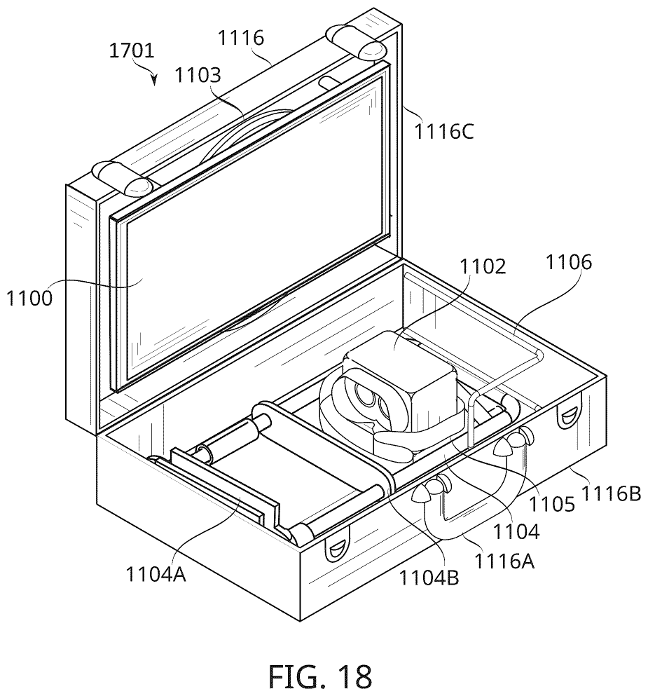

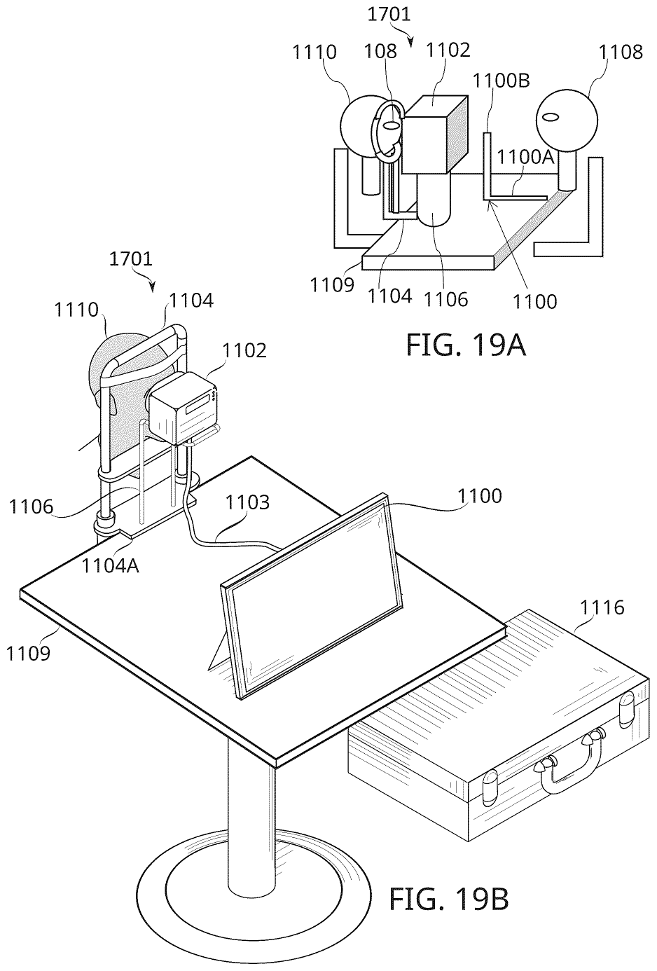

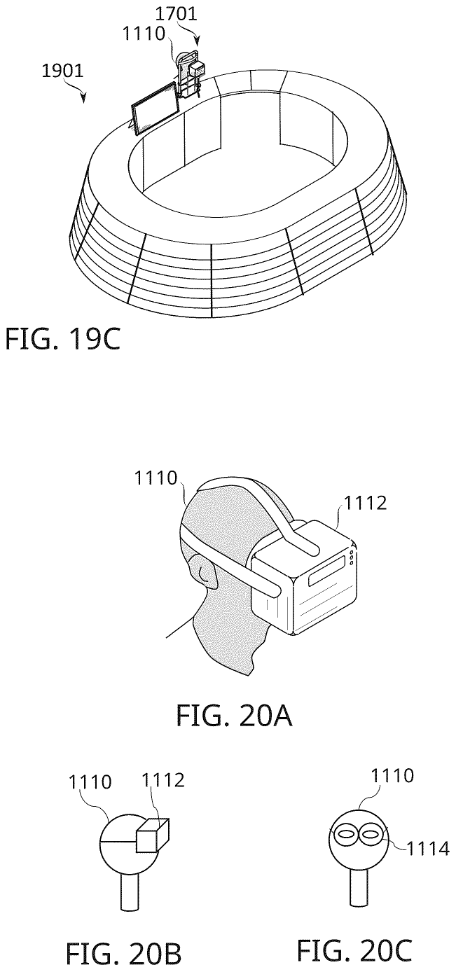

Reference is now made to FIG. 18, which schematically illustrates a compact visual acuity and/or refractive error testing system 1701, according to some embodiments of the present disclosure. Further reference is made to FIGS. 19A-19B, which schematically represent table-top use of compact visual acuity testing kit 1701 to perform visual acuity testing on a subject, according to some embodiments of the present disclosure.

In some embodiments, (for example, as shown in FIG. 18), visual acuity and/or refractive error examination system 1701 comprises controller 1100, optometry apparatus 1102, and mechanical head positioning unit 1104. Optometry apparatus 1102 is shown as approximately head-width, with a somewhat smaller depth and height, but it may optionally be of any size appropriate to containing the testing optics, e.g., with a maximum dimension of 20-50 cm. The testing optics are optionally binocular, or optionally monocular.

Optionally, mechanical connector unit 1106 is provided, configured to support optometry apparatus 1102 above a surface such as a tabletop. Optionally mechanical connector unit 1106 is standalone (e.g., as shown in FIG. 19A). Optionally, it mounts to mechanical head positioning unit 1104, for example, as shown in FIG. 19B.

The schematic view of FIG. 18 illustrates visual acuity and/or refractive error examination system 1701 stored by and/or transferable as a hand-carried unit; e.g., enclosed by a latching case 1116 with base 1116B, lid 1116C, and handle 1116A. Case 1116 may provide internal supports (e.g., foam and/or straps) to hold system elements securely in position (not shown).

Optometry apparatus 1102 comprises a visual acuity and/or refractive error measuring device constructed, for example, according to principles and design elements described in relation to any of the other embodiments described herein; e.g., FIGS. 7-15, optionally operable according any of the methods of FIGS. 16-17, and optionally including features (e.g., cylindrical correction features) as described in relation to any of FIGS. 1A-6.

In some embodiments, controller 1100 comprises input controls 1100A (FIG. 19A) for the examiner 1108 to operate the system, and a display section 1100B upon which the examiner 1108 can observe images, data, procedures, and any other information for conducting visual acuity exams. Optionally, examiner 1108 is present via telecommunications connection, e.g., at an off-site location. Optionally a technician assists positioning the subject, and the examiner 1108 performs the test itself. Arrangements for examiner (test supervisor), and technician (assistant) may correspond, for example, to those described in relation to FIGS. 5 and/or 16.

Optionally, functions for input control and display are combined, e.g., as a touchscreen-equipped controller 1100 (FIGS. 18 and 19B).

To perform visual acuity examinations, optometry apparatus 1102 is aligned with the mechanical head positioning unit 1104. Mechanical head positioning unit 1104 serves to keep the subject's head 1110 in a certain position. It comprises, for example, chin rest 1104B (optionally adjustable in height), forehead strap 1104C, and/or another element which contacts the subject's head to assist in maintaining its position. In particular, head positioning unit 1104 helps to keep the eyes 108 of subject's head 1110 at a certain location during the exams.

In some embodiments, optional mechanical connector unit 1106 supports optometry apparatus 1102 in an appropriate position relative to head positioning unit 1104. It may be free-standing, connected to head positioning unit 1104 (e.g., via clamp 1104A) or otherwise configured to connect or stabilize the apparatus; e.g., to a table 1109 or another surface. Optionally, spatial adjustments (e.g., X, Y, and/or Z displacement) are provided to adjust the position of subject 1110 in head positioning unit 1104, the position of optometry apparatus 1102 as held by mechanical connector unit 1106, and/or clamping position relative to table 1109.

Brief reference is now made to FIG. 19C, which schematically illustrates a refractive error examination system 1701 installed in a kiosk stand 1901. In some embodiments, the apparatus is operated as a service provided as part of a kiosk engaged in selling eyewear and/or optometric services; for example, a space in a shopping mall or other location of commercial (e.g., consumer retail) activity.

For example, in some embodiments, the kiosk is located in a portion of an open retail space, e.g., at least two meters from any wall, open to bypasser foot traffic on two or more sides, and/or disconnected from any wall.

In some embodiments, the kiosk comprises one or more display cabinets. In some embodiments, the kiosk comprises a countertop. Optionally, examination system 1701 is accessible to subjects at the counter top. Optionally, examination system 1701 is accessible to subjects in a space bounded by the counter top, the one or more display cabinets, or any combination thereof.

In some embodiments, subjects place orders for corrective optics, based on a lens prescription obtained through operation of examination system 1701, the order including one or more articles directly displayed and/or exemplified by display in the kiosk (e.g. a glasses frame or portion thereof, a case, or a care accessory).

Reference is now made to FIGS. 20A-20C, which schematically illustrate head-worn implementations of a vision testing system, according to some embodiments of the present disclosure. FIGS. 20A-20B show head-worn embodiments, with optics of the testing system enclosed in housing secured with a strap to the head. FIG. 20C illustrates a more compact embodiment as glasses or goggles. In either case, the optics are optically provided together with other suitable elements of a kit, for example, carrying case 1116, controller 1100, and/or cabling 1103.

### General

It is expected that during the life of a patent maturing from this application many relevant image display types and/or adaptive lens types will be developed; the scopes of the terms “display” and “adaptive lens” are intended to include all such new technologies a priori.

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, to the same extent as if each individual publication, patent or patent application was specifically and individually noted when referenced that it is to be incorporated herein by reference. In addition, citation or identification of any reference in this application shall not be construed as an admission that such reference is available as prior art to the present disclosure. To the extent that section headings are used, they should not be construed as necessarily limiting. In addition, any priority document(s) of this application is/are hereby incorporated herein by reference in its/their entirety.

1-70. (canceled)

71. An image display device, comprising:
an optical pathway configured to project an image onto a retina of an eye, and including cylindrical lenses which introduce varying cylindrical optical power and a varying cylindrical optical axis to beams impinging on the retina to produce the image;
wherein the cylindrical lenses comprise a first cylinder correction lens group and a second cylinder correction lens group, each group comprising at least one cylindrical lens; and
wherein the first and second cylinder correction lens groups:
have cylindrical optical powers of opposite signs;
adjust together to introduce the varying cylindrical axis while maintaining a predetermined relative alignment of their respective cylindrical axes; and
adjust by changing their distance to each other to introduce the varying cylindrical optical power.

72. The image display device of claim 71, wherein the first and second cylinder correction lens groups rotate together around an optical axis of the optical pathway to introduce the varying cylindrical axis.

73. The image display device of claim 72, wherein:
at least the second cylinder correction lens group comprises a plurality of cylindrical lenses; and
cylinder optical powers of the plurality of cylindrical lenses combine along the optical pathway to produce the cylindrical optical power of opposite sign to the first cylinder correction lens group;
wherein the second cylinder correction lens group comprises at least one lens on either side of at least one lens of the first cylinder correction lens group.

74. The image display device of claim 73 wherein at least one lens of the second cylinder correction lens group moves along the optical pathway to vary the introduced cylindrical optical power, and there is at least one position of the at least one lens of the second cylinder correction lens group which cancels cylindrical optical power of the first cylinder correction lens group.

75. The image display device of claim 71, wherein the first and second cylinder correction lens groups are positioned within a telecentric region of the beams.

76. The image display device of claim 71, wherein:
the beams are individually imaged to focal positions on the retina;
each beam having a geometrical center and wherein an envelope defined by encasing the geometrical centers of the beams has an approximately constant size between the first and second cylinder correction lens groups and on a lens group which form an optical pupil, for a range of distances between the first and second cylindrical lens group, corresponding to a range of at least 2 diopters, the lens group which forms the optical pupil is positioned on the optical pathway after the first and second cylindrical correction lens groups.

77. The image display device of claim 71, wherein for the first and second cylinder correction lens groups adjust their distance to each other to change cylindrical correction power through a range of at least 2 diopters.

78. The image display device of claim 71, wherein:
the image display device forms an optical pupil to the eye having a first diameter in a direction wherein the diameter is maximally affected by the varying cylindrical optical power, and a second diameter orthogonal to the first diameter; and
throughout a range of 0 to at least four diopters of adjustment, a ratio of the first and second diameters remains less than 3.

79. The image display device of claim 71, comprising display illumination which generates the image, wherein the display illumination comprises at least one of the group consisting of: a μLED display, a μOLED display, LED display, an OLED display, a QDLED display, an LCD display, and LCOS source, a DLP source, and a scanned beam source.

80. The image display device of claim 73, wherein said at least one lens of the first cylinder correction lens group is moveable between said at least one lens on either side from said second cylinder correction lens group, and along an optical axis of the optical pathway to introduce the varying cylindrical optical power; wherein at least one position of the at least one lens of the first cylinder correction lens group cancels the cylindrical optical power of the second cylinder correction lens group.

81. The image display device of claim 71, wherein the first cylinder correction lens group correspond to a first cylindrical power axis and the second cylinder correction lens group correspond to a second cylindrical power axis and wherein the mutual alignment of the first cylindrical power axis and the second cylindrical power axis is within 5° of each other.

82. An eye examination device comprising the image display device of claim 71, wherein said optical pathway of the image display device is configured to project a subjective eye refraction testing image onto the retina.

83. An eye examination device configured to perform both subjective and objective refraction testing, including, during testing, projection of images and test patterns onto at least a first retina of a tested subject, the eye examination device comprising:
the optical pathway of claim 82;
a second optical pathway, configured to project an objective refraction testing pattern onto the first retina, and comprising a sensor which detects light returning from the first retina from the objective refraction pattern;
wherein the optical pathway projects a target image onto the first retina as a target for visual fixation and/or accommodation control for the subject while the second optical pathway operates to project the objective refraction testing pattern.

84. The eye examination device of claim 83 comprising:
a third optical pathway, configured to merge a view of the surroundings of the tested subject with any of the images and testing pattern of the optical pathway and the second optical pathway.

85. The eye examination device of claim 83, wherein the optical pathway of the image display device configured to project a subjective eye refraction testing image on a retina of a subject is duplicated for producing one or more of:
(a) a subjective eye refraction testing binocularly registered image on each retina of the subject; and
(b) an objective refraction testing pattern binocularly registered image on each retina of the subject.

86. The eye examination device of claim 85, wherein the optical pathway of the image display device configured to project a subjective eye refraction testing image on a retina of a subject is configured to project a sequence of the subjective eye refraction testing binocularly registered images on each retina of the subject.

87. The eye examination device of claim 85, wherein the optical pathway of the image display device configured to project a subjective eye refraction testing image on a retina of a subject is configured to project a sequence of binocularly registered images on each retina of the subject as binocularly registered images on each retina of the subject during the objective refraction testing for fixation and accommodation control.

88. The eye examination device of claim 86, wherein the sequence of subjective eye refraction testing binocularly registered images on each retina of the subject, produces a sequence of perceptually unified images comprising depth cues, such that a perceptually unified image from the sequence of perceptually unified images appears to the subject as having different depth than at least one other perceptually unified image from the sequence of perceptually unified images.

89. The eye examination device of claim 87, wherein the optical pathway of the image display device configured to project a subjective eye refraction testing image sequence of binocularly registered images on each retina of the subject, produces a sequence of perceptually unified images comprising depth cues, such that a perceptually unified image from the sequence of perceptually unified images appears to the subject as having different depth than at least one other perceptually unified image from the sequence of perceptually unified images during the objective refraction testing for fixation and accommodation control.

90. The eye examination device of claim 83, comprising at least one eye tracking device configured to track at least one of eye state and eye position during one or both of subjective eye refraction testing image and objective refraction testing pattern.
